(19) United States
(12) Patent Application Publication
(10) Pub. No.: US 2018/0321258 A1
(43) Pub. Date: Nov. 8, 2018

(54) Exosomal Protein Profiling for Detection of Cardiac Transplant Rejection

(71) Applicant: The Trustees of Columbia University in the City of New York, New York, NY (US)

(72) Inventors: Paul Christian Schulze, New York, NY (US); Peter Kennel, New York, NY (US)

(21) Appl. No.: 15/772,938

(22) PCT Filed: Nov. 7, 2016
(86) PCT No.: PCT/US16/60808

§ 371 Date: May 2, 2018

Related U.S. Application Data
(60) Provisional application No. 62/251,831, filed on Nov. 6, 2015.
(60) Provisional application No. 62/252,537, filed on Nov. 8, 2015.

(51) Int. Cl. G01N 33/68

(52) U.S. Cl. CPC G01N 33/6893; G01N 2800/50; G01N 2800/245

(57) ABSTRACT

The level of exosomal polypeptides in a sample from a patient who has received a transplant is assayed and used as an indicator for transplant rejection. Based on the measured level of the exosomal polypeptides, therapeutic intervention, such as an immunosuppressant therapy, may be started, adjusted, continued or discontinued.

### CROSS REFERENCE TO RELATED APPLICATIONS

This application claims priority to U.S. Provisional Application No. 62/251,831 filed on Nov. 6, 2015, and U.S. Provisional Application No. 62/252,537 filed on Nov. 8, 2015, which are incorporated herein by reference in their entirety.

### STATEMENT REGARDING FEDERALLY SPONSORED RESEARCH OR DEVELOPMENT

This invention was made with government support under grant numbers K23 HL095742-01, P30 HL101272, RO1 HL114813 awarded by the National Institutes of Health. The government may have certain rights in the invention.

### FIELD OF THE INVENTION

The present invention relates to assaying the levels of exosomal proteins/polypeptides for evaluating or monitoring immunological rejection after heart transplant in a patient.

### BACKGROUND OF THE INVENTION

Heart failure (HF) is associated with high morbidity as well as significant mortality. There has been an increased incidence of the disease worldwide. The clinical syndrome of heart failure is the result of heterogeneous myocardial or vascular diseases, and is defined by insufficiency to maintain blood circulation throughout the body. Despite significant advances in the clinical management of HF, conventional therapies are ultimately ineffective in many patients who progress to advanced HF. In these cases, implantation of left ventricular assist devices (LVAD) and/or heart transplantation can be the only viable options.

Heart transplantation (HTx) remains the definitive treatment for severe heart failure. The most common procedure is to take a working heart from a recently deceased organ donor (allograft) and implant it into the recipient. The recipient's heart may either be removed (orthotopic procedure), or less commonly, left in to support the donor heart (heterotopic procedure). Although less successful in comparison to allograft, it is also possible to take a heart from another species (xenograft), or implant a man-made artificial heart. U.S. Patent Application No. 20130209524.

The most common complication of heart transplant is immunological rejection which poses a significant threat to allograft function. Both acute rejection and chronic rejection can occur. Chronic rejection is the major limiting factor for the long-term success of heart transplantation. For example, growth of tissues, such as scar tissue, may cause blockage of the blood vessels of the heart, which ultimately causes the transplanted heart to fail. Two primary causes of graft failure are cell-mediated rejection (CMR) and antibody-mediated rejection (AMR).

Pharmaceutical agents such as cyclosporine A (CSA), steroids and azathioprine are used to control and suppress a recipient's immune system response to grafted tissue. Taylor et al., J. Heart Lung Transplant, 27, 943-956 (2008). Despite universal immunosuppression therapy, rejection is still the principal cause of heart transplant failures. Thus, keeping the immunological rejection to the minimum is a major objective. However, recognizing the onset and severity of rejection is difficult, while the occurrence of rejection is often unpredictable. Tissue rejection in heart transplant recipients is generally silent until the heart is damaged irreversibly. Thus, the transplanted heart tissue must be monitored continuously and carefully for signs of rejection. Early and reliable detection of graft rejection can translate into starting potentially life-saving therapy in time which is vital to the success of heart transplants. Kobashigawa, et al., J. Am. Coll. Cardiol., 45, 1532-1537 (2005).

At present, the only reliable method for monitoring and diagnosing rejection requires frequent endomyocardial biopsy (EMB), an expensive, invasive procedure that must be performed by a specialist. The biopsy is then studied by a pathologist for the invasion of heart tissue by white blood cells, edema, and dead cardiac muscle cells, the histologic manifestations of rejection. EMB is prone to sampling error, the need for repeated, invasive procedures adds significantly to cost and patient discomfort during post-transplant follow-up. Accordingly, there remains a need for a reliable, non-invasive method for detecting rejection.

Recent years have seen growing interest in the identification of biomarkers for the diagnosis and management of various conditions, particularly cancer.⁹⁻¹² Biomarkers serve as reproducible and objective measures of disease state or progression.¹³ Although several recent studies have attempted to identify miRNA- and protein-based serum biomarkers of cardiac allograft rejection, their success has been limited by conflicting data and interindividual variability. Additionally, such approaches typically yield extremely large data sets with high false positive and negative rates.¹⁴⁻¹⁶

Exosomes are small (approximately 30-100 nm) vesicular bodies that are secreted from cells and can enter both neighboring cells and the systemic circulation.¹⁷ Exosomes are actively assembled from intracellular multivesicular bodies (MVBs) by the endosomal sorting complex required for transport (ESCRT) machinery.¹⁸ Based on their cell origin and environment, exosomes can contain specific mRNAs, miRNAs, proteins and lipids.¹⁷ The non-random selection of these contents, which may also be controlled by ESCRT, has led to increasing interest in the role of exosomes in cell-cell signaling, especially in the immune response.^{17,19,20}

In addition to their potential therapeutic applications, exosomes could also represent an entirely new class of biomarkers that are easily detectable in biological fluids and contain only a relatively limited set of biologically active molecules compared to serum.^{19,21} This principle has already guided research into exosome-based biomarkers of several cancers.^{22,23} Identifying changes in serum exosomal protein content in patients experiencing cardiac allograft rejection could therefore offer the possibility of a safer, non-invasive and effective alternative to EMB in the diagnosis of rejection.

This disclosure describes a class of exosomal proteins/polypeptides as biomarkers that allow better diagnostic assessment of patients with rejection following heart transplant or other organ/tissue transplant. The biomarkers also assist in defining the prognosis and the response to treatment.

### SUMMARY

The present invention provides for a method of diagnosing/detecting transplant rejection in a subject (e.g., human) who has received a transplant or a method of assessing the subject's risk of transplant rejection. The method may comprise the steps of: (a) obtaining a sample from the subject (e.g., a plasma, serum or blood sample, or any other sample as discussed herein); (b) isolating exosomes from the sample (e.g., to obtain an exosome preparation); (c) determining/detecting the level of one or more (or 2 or more, 3 or more, 4 or more, 5 or more, 6 or more, 7 or more, 8 or more, 9 or more, 10 or more, 15 or more, or 20 or more) exosomal polypeptides in the exosomes (or in the exosome preparation); (d) comparing the level obtained in step (c) with the level of the one or more exosomal polypeptides in a control sample; and (e) diagnosing that the subject has transplant rejection or an increased risk of transplant rejection, if the level of at least one exosomal polypeptide (or at least 2, at least 3, at least 4, at least 5, at least 6, at least 7, at least 8, at least 9, or at least 10 exosomal polypeptides) obtained in step (c) increases or decreases by at least 10% (or at least 15%, at least 20%, at least 30%, at least 40%, at least 50%, at least 60%, at least 70%, at least 80%, at least 90%, about 20% to about 90%, about 50% to about 100%, at least 1 fold, at least 1.5 fold, at least 2 fold, at least 2.5 fold, or at least 3 fold) compared to its level in the control sample.

Also encompassed by the present invention is a method of treating a subject (e.g., human) with transplant rejection or an increased risk of transplant rejection (and/or treating a subject predicted to undergo transplant rejection). The method may comprise the steps of: (a) obtaining a sample from the subject (e.g., a plasma, serum or blood sample, or any other sample as discussed herein); (b) isolating exosomes from the sample (e.g., to obtain an exosome preparation); (c) determining/detecting the level of one or more (or 2 or more, 3 or more, 4 or more, 5 or more, 6 or more, 7 or more, 8 or more, 9 or more, 10 or more, 15 or more, or 20 or more) exosomal polypeptides in the exosomes (or in the exosome preparation); (d) comparing the level obtained in step (c) with the level of the one or more exosomal polypeptide in a control sample; and (e) treating the subject for transplant rejection or an increased risk of transplant rejection, if the level of at least one exosomal polypeptide (or at least 2, at least 3, at least 4, at least 5, at least 6, at least 7, at least 8, at least 9, or at least 10 exosomal polypeptides) obtained in step (c) increases or decreases by at least 10% (or at least 15%, at least 20%, at least 30%, at least 40%, at least 50%, at least 60%, at least 70%, at least 80%, at least 90%, about 20% to about 90, about 50% to about 100%, at least 1 fold, at least 1.5 fold, at least 2 fold, at least 2.5 fold, or at least 3 fold) compared to its level in the control sample.

The present invention provides for a method of detecting transplant rejection in a subject (e.g., human) who has received a transplant or assessing the subject's risk of transplant rejection. The method may comprise the steps of: (a) obtaining a sample from the subject (e.g., a plasma, serum or blood sample, or any other sample as discussed herein); (b) determining in the sample the level of one or more (or 2 or more, 3 or more, 4 or more, 5 or more, 6 or more, 7 or more, 8 or more, 9 or more, 10 or more, or 15 or more) polypeptides selected from the group consisting of C1QA, C1R, KV302, HV304, HV315, FIBA, FIBB, FIBG, FINC, F13A, TSP1, FRMPD1, IT1H, APOL1 and ACTB; (c) comparing the level obtained in step (b) with the level of the one or more polypeptides in a control sample; and (d) diagnosing that the subject has transplant rejection or an increased risk of transplant rejection, if the level of at least one polypeptide (or at least 2, at least 3, at least 4, at least 5, at least 6, at least 7, at least 8, at least 9, or at least 10 exosomal polypeptides) obtained in step (b) increases or decreases by at least 10% (or at least 15%, at least 20%, at least 300%, at least 40%, at least 50%, at least 60%, at least 70%, at least 80%, at least 90%, about 20% to about 90%, about 50% to about 100%, at least 1 fold, at least 1.5 fold, at least 2 fold, at least 2.5 fold, or at least 3 fold) compared to its level in the control sample. In certain embodiments, the polypeptide is an exosomal protein/polypeptide. In certain embodiments, after step (a) exosomes are isolated from the sample (e.g., to obtain an exosome preparation), and in step (b) the level of the at least one polypeptide in the exosomes (or in the exosome preparation) is determined.

The present invention also provides for a method of treating a subject (e.g., human) with transplant rejection or an increased risk of transplant rejection. The method may comprise the steps of: (a) obtaining a sample from the subject (e.g., a plasma, serum or blood sample, or any other sample as discussed herein); (b) determining in the sample the level of one or more (or 2 or more, 3 or more, 4 or more, 5 or more, 6 or more, 7 or more, 8 or more, 9 or more, 10 or more, or 15 or more) polypeptide selected from the group consisting of C1QA, C1R, KV302. HV304, HV315, FIBA, FIBB, FIBG, FINC, F13A, TSP1, FRMPD1, IT1H1, APOL1 and ACTB; (c) comparing the level obtained in step (b) with the level of the one or more polypeptide in a control sample; and (d) treating the subject for transplant rejection or an increased risk of transplant rejection, if the level of at least one polypeptide (or at least 2, at least 3, at least 4, at least 5, at least 6, at least 7, at least 8, at least 9, or at least 10 exosomal polypeptides) obtained in step (b) increases or decreases by at least 10% (or at least 15%, at least 20%, at least 30%, at least 40%, at least 50%, at least 60%, at least 70%, at least 80%, at least 90%, about 20% to about 90%, about 50% to about 100%, at least 1 fold, at least 1.5 fold, at least 2 fold, at least 2.5 fold, or at least 3 fold) compared to its level in the control sample. In certain embodiments, the polypeptide is an exosomal protein. In certain embodiments, after step (a) exosomes are isolated from the sample (e.g., to obtain an exosome preparation), and in step (b) the level of the at least one polypeptide in the exosomes (or in the exosome preparation) is determined.

The transplant can be a heart transplant, a kidney transplant, a pancreas transplant, a liver transplant, a lung transplant, an intestine transplant, or a combination thereof.

In certain embodiments, the transplant is a tissue transplant or an organ transplant.

In certain embodiments, the exosomal polypeptide detected/determined may be C1QA, C1R, KV302, HV304, HV315, FIBA, FIBB, FIBG, FINC, F13A, TSP1, FRMPD1, IT1H1, APOL1, ACTB, or combinations thereof.

In certain embodiments, the exosomal polypeptide detected/determined may be C1QA, FINC, KV302, HV304, or combinations thereof.

In certain embodiments, the exosomal polypeptide detected/determined may be LV101, IGJ, STK36, L1CAM, KV302, ITIH2, PLMN, PON1, C1RL, KV303, KV1A1, B7ZKJS, FIBG, FIBB, CO5, LV102, A2AP, or combinations thereof.

In certain embodiments, the exosomal polypeptide detected/determined may be LV02, FIBG, FIBB, FIBA, ACTB, ECM1, F13A, C1R, FINC, TSP1, TNNC1, FSVV04, STK36, IGJ, TOP2A, LV101, TRIPB, GK, LICAM, PON1, C1RL, ITIH2, KLKB1, HV315, APOL1, GELS, IGHD, IT1H1, FRMPD1, PLMN, KV302, FSW6P5, C9JMH6, B7ZKJS, KV1A1, F5H7E1, A1AG1, A2AP, HV304, GSJLSS, E9PBC5, QSVY30, Q5T9S5, C9JA05, F5H4W9, or combinations thereof.

In certain embodiments, the exosomal polypeptide detected/determined may be fibronectin, IGHM, LV101, HBB, or combinations thereof.

In certain embodiments, the exosomal polypeptide detected/determined may be one or more selected from the exosomal polypeptides/proteins listed in Table 1.

In certain embodiments, the subject is treated with an immunosuppressant.

In certain embodiments, the subject's existing immunosuppressive regimen is modified or maintained.

The level of the one or more polypeptides may be determined/detected by mass spectrometry (MS), and/or enzyme-linked immunosorbent assay (ELISA).

The control sample may be from a subject who has received a transplant without rejection or from a plurality of subjects who have received a transplant without rejection. The control sample may be from a healthy subject or from a plurality of healthy subjects.

In certain embodiments, the transplant rejection comprises acute cellular rejection (ACR) and/or antibody-mediated rejection (AMR).

In certain embodiments, the transplant rejection comprises hyperacute rejection.

In certain embodiments, the transplant rejection comprises acute rejection.

In certain embodiments, the transplant rejection comprises chronic transplant rejection.

The present invention also provides for a kit comprising: antibodies or fragments thereof that specifically bind to one or more exosomal polypeptides in a plasma, serum or blood sample from a subject who has received a transplant; and instructions for measuring the one or more exosomal polypeptides for diagnosing transplant rejection in the subject or assessing the subject's risk of transplant rejection.

### BRIEF DESCRIPTION OF THE FIGURES

FIG. 1A is a heatmap showing that exosomal protein profiling distinguishes between various cardiac pathologies. A total of 45 proteins were identified that could distinguish at least one group from the rest of the dataset at q<0.05.

FIG. 1B. Principal component analysis (PCA) demonstrates 3 distinct groupings of exosomal protein signatures correlating with patient phenotype: (1) control and HF: (2) HTx, no rejection; and (3) ACR and AMR.

FIG. 2A. HF and HTx are associated with distinct changes in exosomal proteins relative to controls. A total of 17 proteins were identified that could distinguish at least one group at q<0.05.

FIG. 2B. PCA reveals distinct groupings for the data from each cohort.

FIG. 3A. Limma empirical Bayes analysis of serum exosomal protein counts in non-rejection HTx, ACR and AMR samples identified 15 proteins that could distinguish at least one group from the dataset at q<0.05.

FIG. 3B. PCA shows each of the 3 cohorts forming a distinct data cluster. Of these 15 proteins, 8 are associated with immunological processes.

FIG. 4. Healthy control vs. heart failure. Exosomal protein content analysis revealed only minor differences in protein signatures in the control versus heart failure comparison. Two group t-test was applied to the selected dataset. A potential outliner (Csm10) was identified and removed to generate a protein signature. The filtering criteria were set at p<0.01 and a suggested variance of 0.21 (σ/σmax) to render clustering of biological replicates within each group. The corresponding q-value is 0.19 (˜18%, FDR). This filtering criteria yielded a signature of 7 proteins.

FIG. 5. Health control vs. HTx no rejection. Analysis of rejection samples without evidence of rejection, confirmed by pathology report, revealed significant changes in immunoglobulin subfractions such as KV303 and LV106 as well as fibrinogen components. Two group t-test was applied to the selected dataset. The filtering criteria were set at q<0.05 (5% FDR) and suggested variance of 0.06 (σ/σmax) to render clustering of biological replicates within each group. The adjusted p-value is 0.0028. This filtering criteria yielded a protein signature of 16 proteins. It appears that there are two subgroups within the HTX no rejection group.

FIGS. 6A-6C. HTx no Rejection vs. rejection (ACR and AMR). Cohort comparison of no rejection vs rejection (ACR and AMR) patients yielded differences in exosomal protein content of proteins related to immune mechanisms. (A) Principle component analysis. (B) Heatmap. (C) Number of hits of FINC, D6R934 (C1 component), F13A, C1Q, IGHM and FIBA in no rejection, AMR and ACR samples. Of note, complement factor components such as C1 fractions, which are known to initiate the classical pathway of the complement system are strongly decreased in the rejection cohort. Also, fibronectin is significantly decreased, a protein which has been linked to acute and chronic transplant rejection. Furthermore, antibody fractions such as IGHM and LV101 were altered in the rejection group. Two group t-test was applied to the selected dataset. The filtering criteria were set at q<0.02 (2% FDR) and a suggested variance of 0.0186 (σ/σmax) to render clustering of biological replicates within each group. The adjusted p-value is 7.6576e-4. This filtering criteria yielded a protein signature of 24 proteins.

FIG. 7. HTx no rejection vs. ACR. Sub-cohort analysis of ACR revealed differences in exosomal protein content of proteins related to immune responses such as fibrinogen components, complement factors such as C1 components and Ig fractions. Two group t-test was applied to the selected dataset. The filtering criteria were set at q<0.05 (5% FDR) and suggested variance of 0.06 (σ/σmax) to render clustering of biological replicates within each group. The adjusted p-value is 0.0028. This filtering criteria yielded a protein signature of 16 proteins. It appears that there are two subgroups within the HTX no rejection group when comparing the HTX no rejection patients with ACR rejection patients.

FIG. 8. HTx no rejection vs. AMR. Sub-cohort analysis of AMR-type rejection revealed differences in exosomal protein content of proteins related to an immune response such as fibrinogen components, complement factors and Ig fractions. However, no signature could be derived to differentiate between ACR and AMR-type rejection. Two group t-test was applied to the selected dataset. The filtering criteria were set at q<0.0S (5% FDR) and suggested variance of 0.05 (σ/σmax) to render clustering of biological replicates within each group. The adjusted p-value is 0.0017. This filtering criteria yielded a protein signature of 13 proteins.

### DETAILED DESCRIPTION

The methods of the present disclosure assay the levels of exosomal proteins/polypeptides in a sample (e.g., a plasma or serum sample) taken from a patient who has received a transplant, such as a heart transplant. The levels of exosomal proteins/polypeptides in the sample can be used for assessing the onset or severity of transplant rejection, or as an indicator of the efficacy of a therapeutic intervention for treating transplant rejection. A plurality of exosomal proteins/polypeptides may be measured. Based on the levels of the exosomal proteins/polypeptides, transplant rejection may be diagnosed or predicted, and then the subject may be treated. For patients under an immunosuppressive therapy, based on the exosomal protein/polypeptide levels, the therapeutic intervention may be continued when it is effective, or altered if ineffective or insufficient.

The method may also identify a transplant recipient at risk for transplant rejection or delayed graft function. As such, the methods of the present disclosure can impact the way transplant recipients are treated (before, during, and/or after a transplantation procedure). For example, patients identified as having a high risk of transplant rejection can be treated more aggressively with, for example, immunosuppressants or other therapeutic agents. Patients identified as low risk may be treated less aggressively (e.g., with minimal or no immunosuppressants).

The present methods can diagnose or predict transplant rejection in a subject who has received a transplant.

In certain embodiments, the method contains the following steps: (a) obtaining a sample (e.g., a plasma or serum sample, or other samples as discussed herein) from the subject; (b) assaying the level of one or more exosomal proteins/polypeptides in the sample; and (c) comparing the level obtained in step (b) with the level of the one or more exosomal proteins/polypeptides in a control sample. The subject is diagnosed to undergo transplant rejection (or diagnosed to have an increased risk of transplant rejection), if the level of at least one exosomal protein/polypeptide obtained in step (b) increases or decreases by at least 5% compared to its level in the control sample.

The present methods may treat a subject with transplant rejection or an increased risk of transplant rejection. When diagnosed with transplant rejection, the subject may be treated with at least one immunosuppressant. Alternatively, when transplant rejection is predicted (or when an increased risk of transplant rejection is diagnosed), the subject may be treated with at least one immunosuppressant.

In certain embodiments, the method contains the following steps: (a) obtaining a sample (e.g., a plasma or serum sample, or other samples as discussed herein) from the subject; (b) assaying the level of one or more exosomal proteins/polypeptides in the sample; (c) comparing the level obtained in step (b) with the level of the one or more exosomal proteins/polypeptides in a control sample; and (d) treating the subject for transplant rejection or an increased risk of transplant rejection, if the level of at least one exosomal protein/polypeptide obtained in step (b) increases or decreases by at least 5% compared to its level in the control sample.

In certain embodiments, the present method determines/detects the level of one or more exosomal polypeptides selected from C1QA, C1R, KV302, HV304, HV315, FIBA, FIBB, FIBG, FINC, F13A, TSP1, FRMPD1, ITIH1, APOL1, ACTB, and combinations thereof.

In certain embodiments, the present method determines/detects the level of one or more exosomal polypeptides selected from C1QA, FINC, KV302, HV304, and combinations thereof.

In certain embodiments, the present method determines/detects the level of one or more exosomal polypeptides selected from LV101, IGJ, STK36, LICAM, KV302, ITIH2, PLMN, PON1, C1RL, KV303, KV1A1, B7ZKJS, FIBG, FIBB, CO5, LV102, A2AP, and combinations thereof.

In certain embodiments, the present method determines/detects the level of one or more exosomal polypeptides selected from LV102, FIBG, FIBB, FIBA, ACTB, ECM1, F13A, C1R, FINC, TSP1, TNNC1, FSVV04, STK36, IGJ, TOP2A, LV101, TRIPB, GK, LICAM, PON1, C1RL, ITIH2, KLKB1, HV315, APOL1, GELS, IGHD, IT1H1, FRMPD1, PLMN, KV302, KV303, CO5, C1QA, FSW6P5, C9JMH6, B7ZKJS, KV1A1, F5H7E1, A1AG1, A2AP, HV304, GSJLSS, E9PBC5, QSVY30, Q5T9S5, C9JA05, F5H4W9, and combinations thereof.

In certain embodiments, the present method determines/detects the level of one or more exosomal polypeptides selected from fibronectin, IGHM, LV101, HBB, and combinations thereof.

In certain embodiments, the present method determines/detects the level of one or more exosomal polypeptides selected from those listed in Table 1, and combinations thereof.

Table 1 provides an exemplary list of exosomal proteins/polypeptides whose levels may be determined/detected by the present method. There may be a number of different isoforms for each of these exosomal proteins/polypeptides, provided herein are the general accession numbers, NCBI Reference Sequence (RefSeq) accession numbers, GenBank accession numbers, and/or UniProt numbers to provide relevant sequences. The exosomal proteins/polypeptides may also comprise other sequences.

TABLE 1
List of Selected Exosomal Proteins

| Protein | Protein Accession |
|---|---|
| FRMPD1 | NP_055722 |
| (FERM and PDZ domain-containing | |
| protein 1) | |
| ACTB | NP_001092 |
| (actin, cytoplasmic 1) | |
| FIBG | P02679 |
| (Fibrinogen gamma chain) | |
| FIBB | P02675 |
| (Fibrinogen beta chain) | |
| FIBA | P02671 |
| (Fibrinogen alpha chain) | |
| TSP1 | P07996 |
| (Thrombospondin-1) | |
| FINC | |
| C1R | NP_001724 |
| (complement C1r subcomponent | |
| precursor) | |
| C1QA | P02745 |
| (Complement C1q subcomponent | |
| subunit A; precursor) | |
| F13A | NP_000120; P00488 |
| (coagulation factor XIII A | |
| chain precursor) | |
| LV101 | — |
| LV102 | P01700 |
| ECM1 | Q16610 |
| (Extracellular matrix protein 1) | |
| TNNC1 | P63316 |
| (Troponin C, slow skeletal and | |
| cardiac muscles) | |
| FSVV04 | |
| STK36 | Q9NRP7 |
| (Serine/threonine-protein kinase 36) | |
| IGJ | P01591 |
| (Immunoglobulin J chain) | |
| TOP2A | P11388 |
| (DNA topoisomerase 2-alpha) | |
| TRIPB | Q15643 |
| (Thyroid receptor-interacting | |
| protein 11) | |
| GK | P32189 |
| (Glycerol kinase) | |
| L1CAM | P32004 |
| (Neural cell adhesion molecule L1_) | |
| PON1 | P27169 |
| (Serum paraaxonase/arylesterase 1) | |
| C1RL | Q9NZP8 |
| (Complement C1r subcomponent-like | |
| protein) | |
| ITIH1 | EAW65259 |
| (inter-alpha (globulin) inhibitor H1) | |
| ITIH2 | P19823 |
| (Inter-alpha-trypsin inhibitor | |
| heavy chain H2) | |
| KLKB1 | P03952 |
| (Plasma kallikrein) | |
| HV315 | P01776 |
| APOL1 | AAI41824; O14791 |
| (Apolipoprotein L1) | |
| GELS | P06396 |
| (Gelsolin) | |
| IGHD | P01880 |
| (Ig delta chain C region) | |
| PLMN | P00747 |
| (Plasminogen) | |
| KV302 | — |
| FSW6P5 | — |
| C9JMH6 | alpha-2-antiplasmin |
| | accession number: P08697 |
| B7ZKJS | — |
| KV1A1 | P01632 |
| (Ig kappa chain V-I region S107A) | |
| F5H7E1 | — |
| A1AG1 | P02763 |
| (Alpha-1-acid glycoprotein 1) | |
| A2AP | P08697 |
| Alpha-2-antiplasmin | |
| HV304 | — |
| GSJLSS | — |
| E9PBC5 | E9PBC5; |
| (Plasma kallikrein) | P03952 |
| Q5VY30 | Q5VY30 |
| (Retinol binding protein 4, | |
| plasma, isoform CRA_b) | |
| Q5T9S5 | Q5T9S5 |
| (Coiled-coil domain-containing | |
| protein 18) | |
| C9JA05 | C9JA05 |
| (Immunoglobulin J chain) | (Full length protein |
| | accession: P01591) |
| F5H4W9 | U6DUW6 |
| (Paraoxonase 1) | |

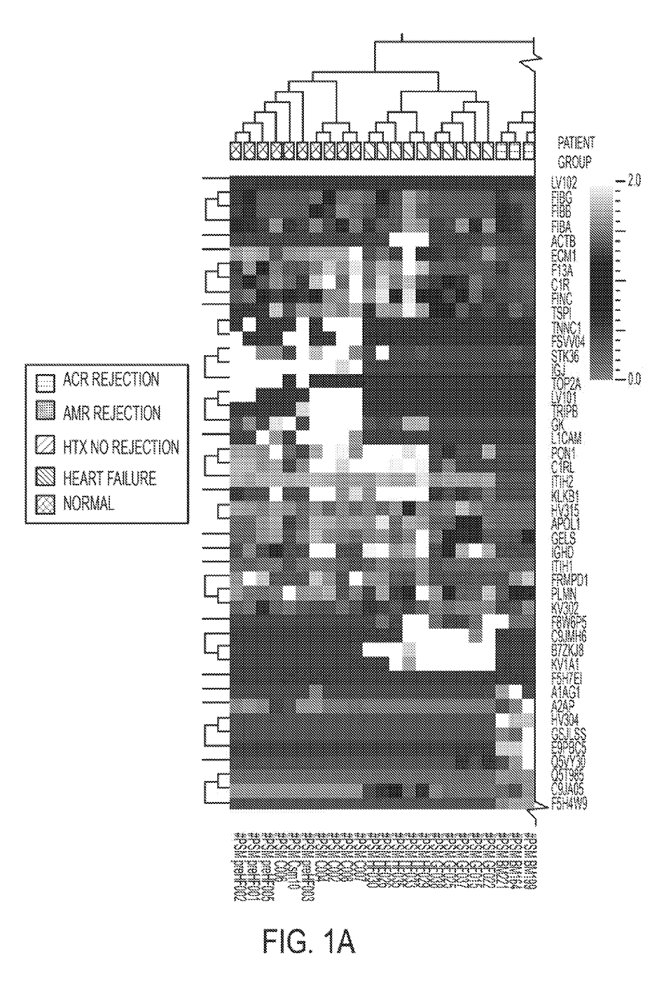

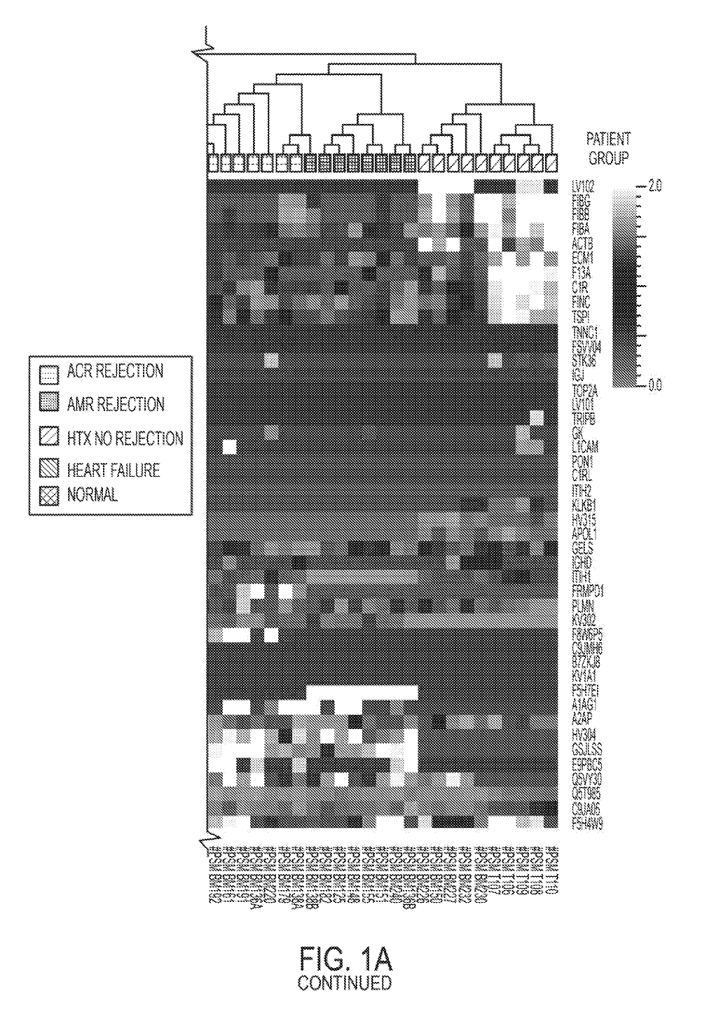

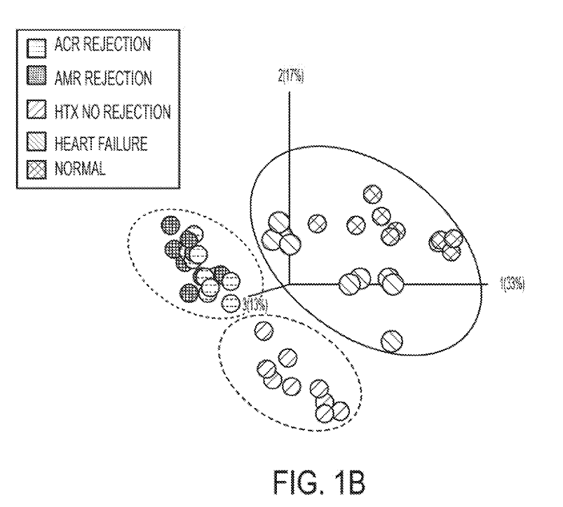

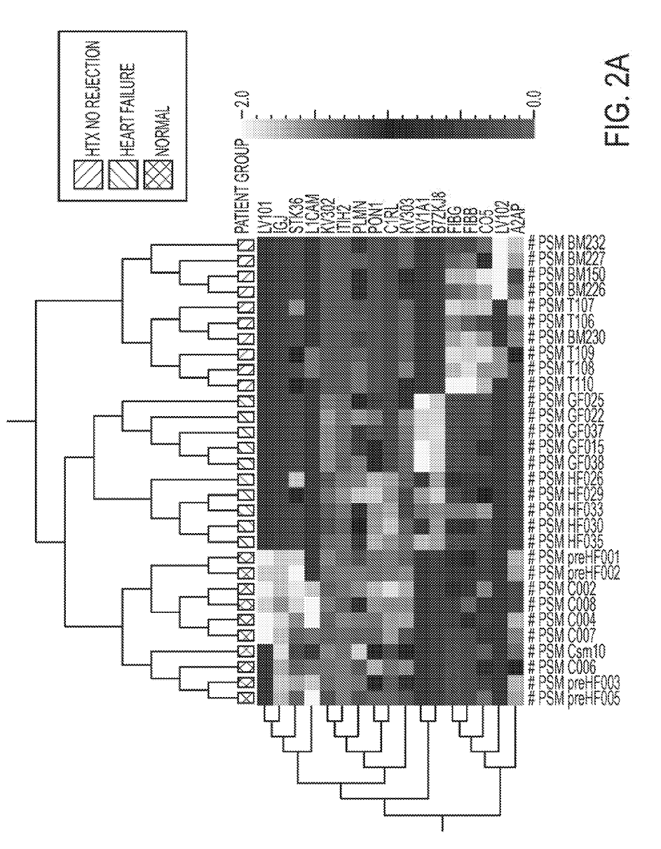

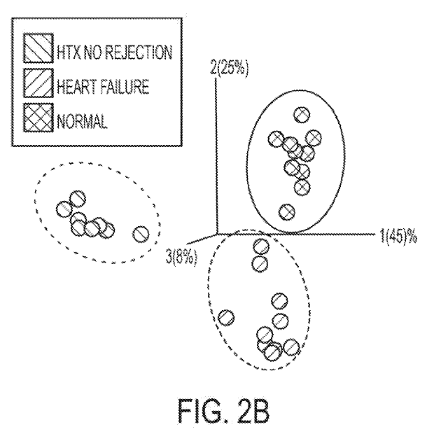

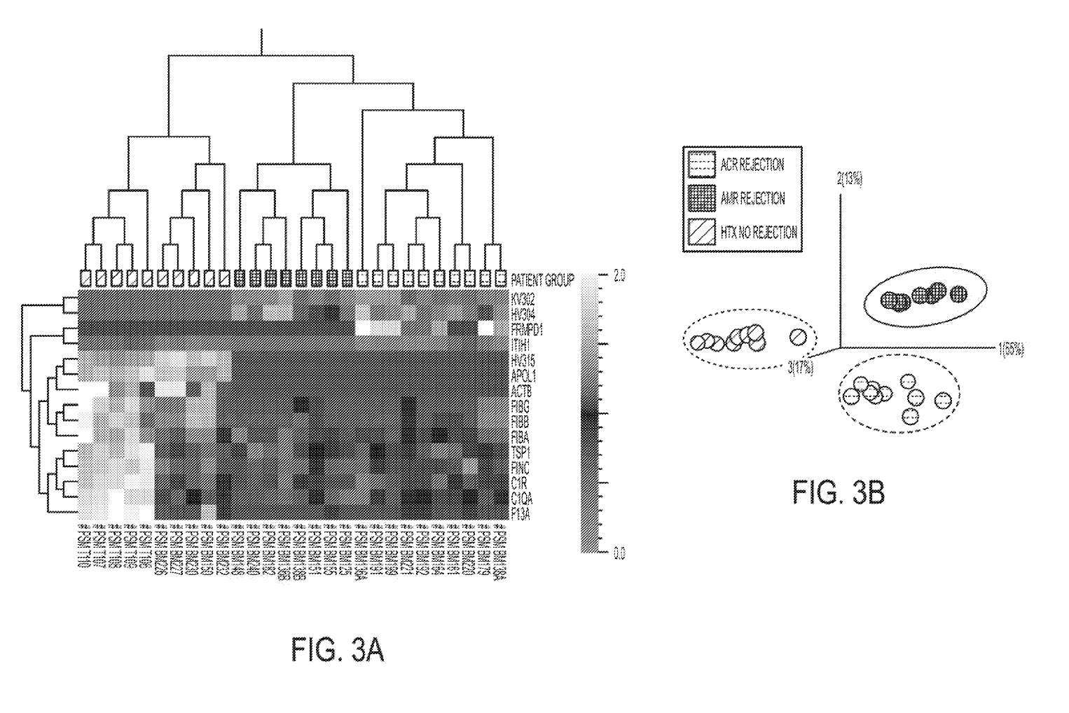

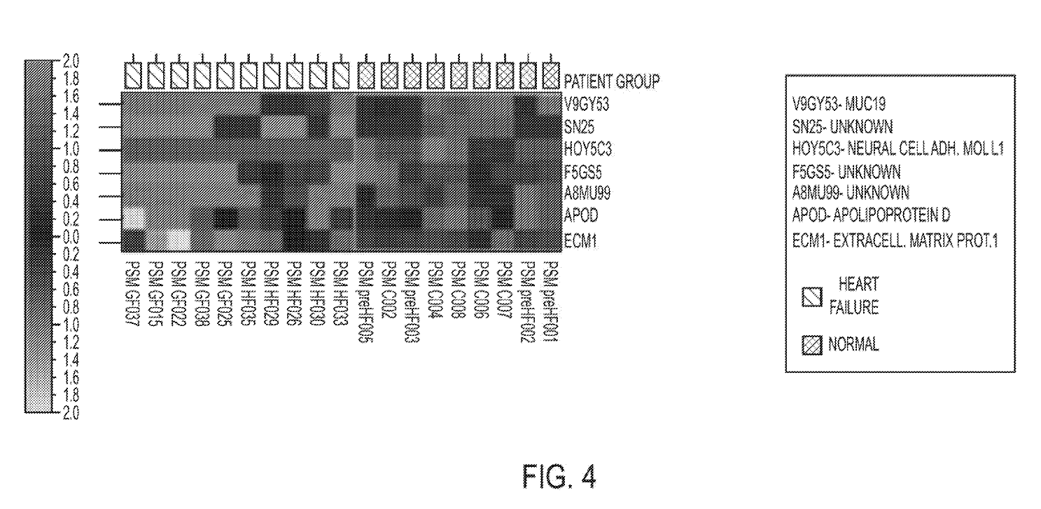

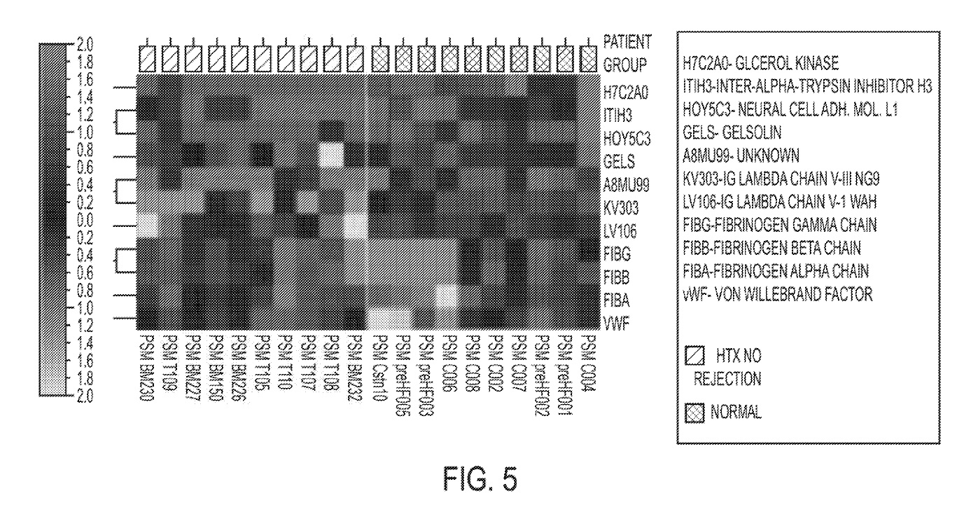

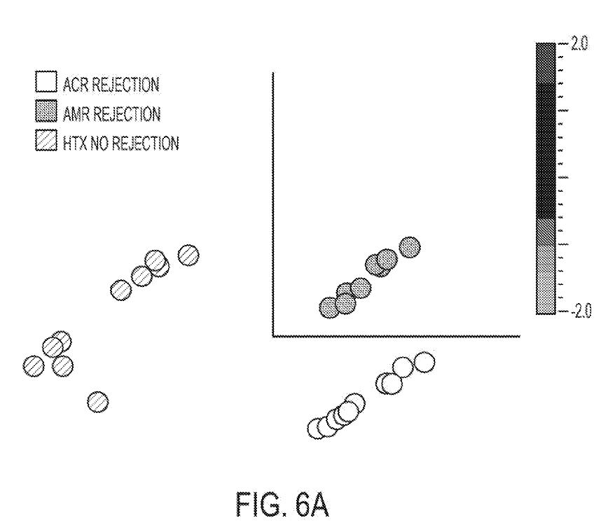

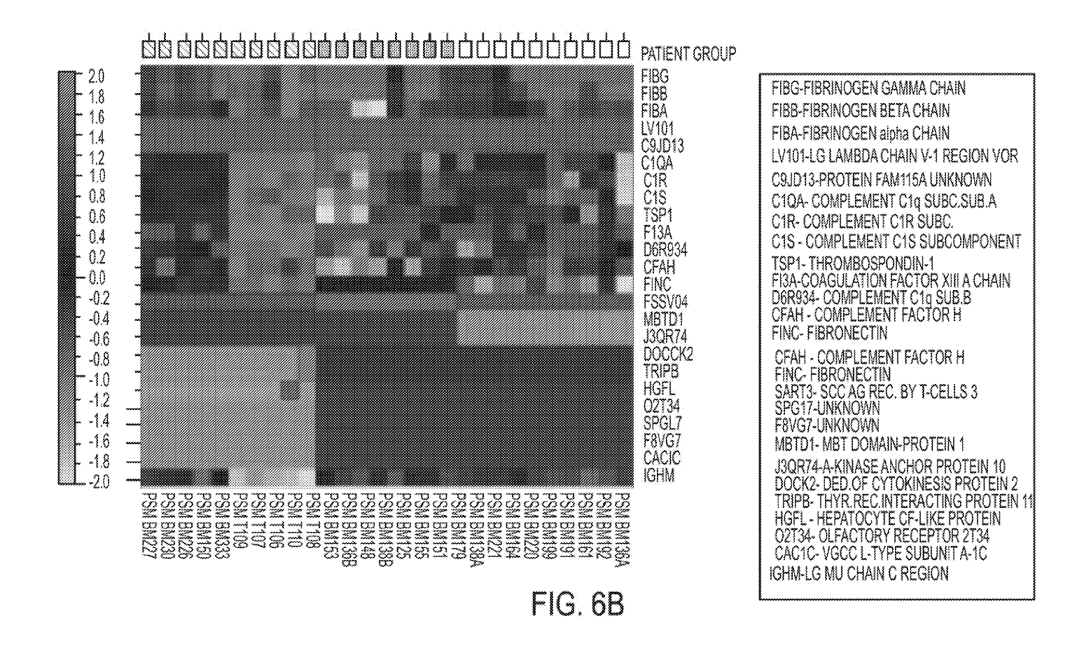

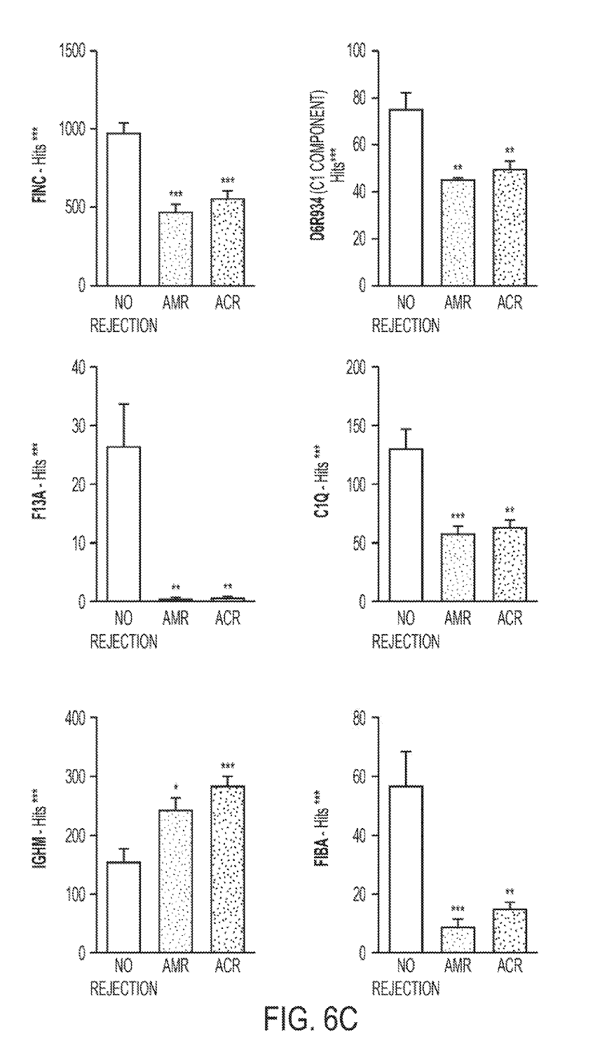

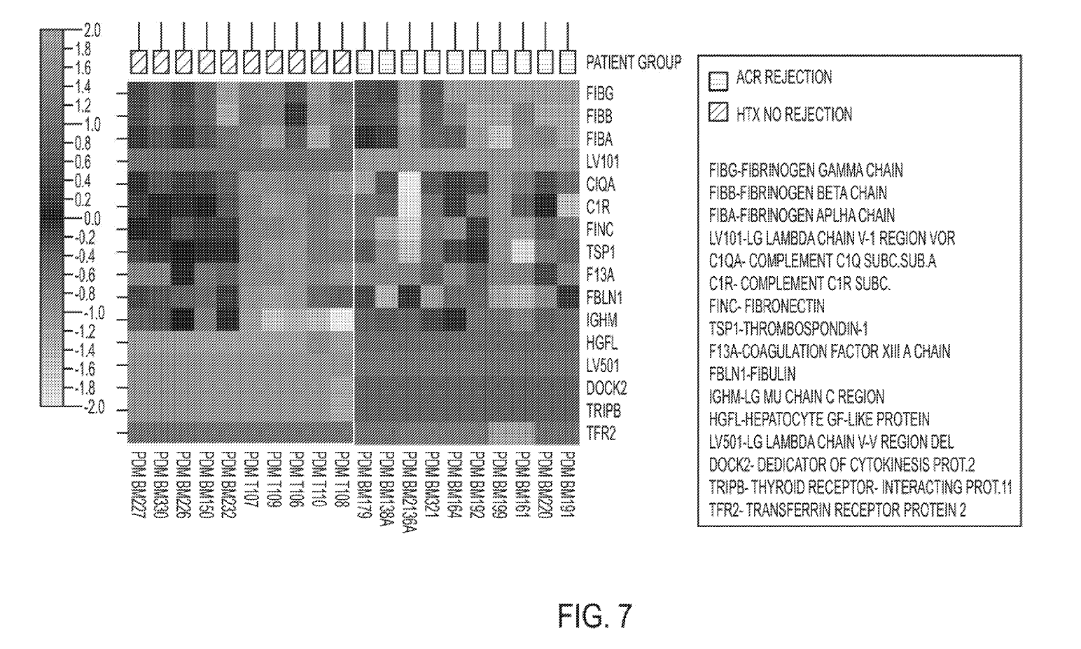

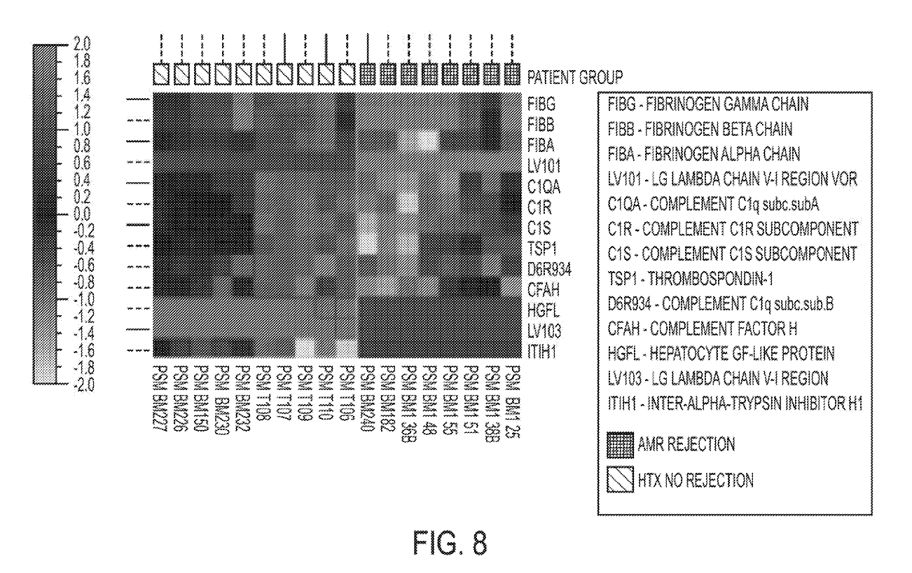

In certain embodiments, the present method determines/detects the level of one or more exosomal polypeptides selected from the exosomal polypeptides/proteins in FIG. 1A, FIG. 2A, FIG. 3A, FIG. 4, FIG. 5, FIG. 6B, FIG. 6C, FIG. 7. FIG. 8, and combinations thereof.

In certain embodiments, the method contains the following steps: (a) obtaining a sample from the subject; (b) determining (detecting) in the sample a level of expression of one or more polypeptides selected from C1QA, C1R, KV302, HV304, HV315, FIBA, FIBB, FIBG, FINC, F13A, TSP1, FRMPD1, IT1H1, APOL1, ACTB, LV101, C1Q, HBB, IGJ, STK36, LICAM, ITIH2, PLMN, PON1, C1RL, KV303, KV1A1, B7ZKJS, CO5, LV102, A2AP, ECM1, TNNC1, FSVV04, TOP2A, TRIPB, GK, KLKB1, GELS, IGHD, F8W6P5, C9JMH6, F5H7E1, A1AG1, G8JL88, E9PBC5, Q5VY30, Q5T985, C9JA05, and F5H4W9, wherein an increase or decrease by at least 5% in the level of the one or more polypeptides relative to a control sample indicates that the subject has transplant rejection or have an increased risk of transplant rejection.

The level of at least one, or at least 2 (or at least 3, at least 4, at least 5, at least 6, at least 7, at least 8, at least 9, at least 10, at least 20, at least 30, at least 40, between 5 and 30, between 5 and 10, between 2 and 6, between 3 and 5, between 10 and 20, or between 20 and 45) exosomal proteins/polypeptides in the sample may increase or decrease by about 1% to about 100%, about 5% to about 90%, about 10% to about 80%, about 5% to about 70%, about 5% to about 60%, about 10% to about 50%, about 15% to about 40%, about 5% to about 20%, about 1% to about 20%, about 10% to about 30%, at least about 5%, at least about 10%, at least about 15%, at least about 20%0, at least about 30%, at least about 40%, at least about 50%, at least about 60%, at least about 70%, at least about 80%, at least about 90%, at least about 100%, about 10% to about 90%, about 12.5% to about 800%, about 20% to about 70%, about 25% to about 60%, or about 25% to about 50%, about 2 fold, about 3 fold, about 4 fold, about 5 fold, about 6 fold, about 7 fold, about 8 fold, about 9 fold, about 10 fold, at least 1.1 fold, at least 1.2 fold, at least 1.3 fold, at least 1.4 fold, at least 1.5 fold, at least 1.6 fold, at least 1.8 fold, at least 2 fold, at least 2.5 fold, at least 3 fold, at least 3.5 fold, at least 5 fold, at least 10 fold, at least 15 fold, at least 20 fold, at least 50 fold, at least 100 fold, at least 120 fold, from about 2 fold to about 500 fold, from about 1.1 fold to about 10 fold, from about 1.1 fold to about 5 fold, from about 1.5 fold to about 5 fold, from about 2 fold to about 5 fold, from about 3 fold to about 4 fold, from about 5 fold to about 10 fold, from about 5 fold to about 200 fold, from about 10 fold to about 150 fold, from about 10 fold to about 20 fold, from about 20 fold to about 150 fold, from about 20 fold to about 50 fold, from about 30 fold to about 150 fold, from about 50 fold to about 100 fold, from about 70 fold to about 150 fold, from about 100 fold to about 150 fold, from about 10 fold to about 100 fold, from about 100 fold to about 200 fold, compared to the level(s) in the control sample. The control sample may be from a patient who has received a transplant without rejection or a plurality of patients who have received a transplant without rejection. The control sample may be from a healthy subject or a plurality of healthy subjects.

In certain embodiments, the levels of a plurality of exosomal proteins/polypeptides in the sample may be assayed, which comprises 2 or more, 3 or more, 4 or more, 5 or more, 6 or more, 7 or more, 8 or more, 9 or more, 10 or more, 15 or more, 20 or more, 25 or more, 30 or more, 35 or more, 3-50, 5-50, 10-50, 15-50, 20-50, 30-50, or 50-100, exosomal proteins/polypeptides.

The samples may include, but are not limited to, serum, plasma, blood, whole blood and derivatives thereof, cardiac tissue, bone marrow, urine, cerebrospinal fluid (CSF), myocardium, endothelium, skin, hair, hair follicles, saliva, oral mucus, vaginal mucus, sweat, tears, epithelial tissues, semen, seminal plasma, prostatic fluid, excreta, ascites, lymph, bile, as well as other samples or biopsies. In one embodiment, the biological sample is plasma or serum.

The level or amount of a polypeptide in a patient sample can be compared to a reference level or amount of the polypeptide present in a control sample. The control sample may be from a patient or patients with a cardiovascular disease (e.g., heart failure) or a healthy subject or subjects. In other embodiments, a control sample is taken from a patient prior to transplant or treatment with a therapeutic intervention, or a sample taken from an untreated patient. In certain embodiments, a control sample is from transplant recipients without transplant rejection. Reference levels for a polypeptide can be determined by determining the level of a polypeptide in a sufficiently large number of samples obtained from normal, healthy control subjects to obtain a pre-determined reference or threshold value. A reference level can also be determined by determining the level of the polypeptide in a sample from a patient prior to transplant. Reference (or calibrator) level information and methods for determining reference levels can be obtained from publicly available databases, as well as other sources.

The transplant may be an allograft or a xenograft. An allograft is a transplant of an organ, tissue, bodily fluid or cell from one individual to a genetically non-identical individual of the same species. A xenograft is a transplant of an organ, tissue, bodily fluid or cell from a different species.

The transplant maybe any organ or tissue transplant, including, but not limited to, a heart transplant, a kidney transplant, a liver transplant, a pancreas transplant, a lung transplant, an intestine transplant, a skin transplant, a bone marrow transplant, a small bowel transplant, a trachea transplant, a cornea transplant, a limb transplant, and a combination thereof.

The present methods may diagnose or predict any type of transplant rejection, including, but not limited to, hyperacute rejection, acute rejection, and/or chronic rejection.

The present methods may determine/detect the presence, type and/or severity of the transplant rejection.

Also encompassed by the present disclosure is a method for assessing efficacy of an immunosuppressant therapy for transplant rejection in a patient. The method may contain the following steps: (a) obtaining a first sample from the patient before initiation of the therapy (or at a first time point after initiation of the therapy); (b) assaying the levels of one or more exosomal proteins/polypeptides in the first sample; (c) obtaining a second sample from the patient after initiation of the therapy (or at a second time point after initiation of the therapy); (d) assaying the levels of the one or more exosomal proteins/polypeptides in the second sample; (e) comparing the levels of step (b) with the levels of step (d). If the level of at least one, or at least 2 (at least 3, at least 4, at least 5, at least 6, at least 7, at least 8, at least 9, at least 10, at least 20, at least 30, at least 40, at least 50, between 5 and 30, between 5 and 10, between 10 and 20, between 30 and 50, or between 50 and 100) exosomal proteins/polypeptides obtained in step (d) increases or decreases by about 1% to about 10%, about 5% to about 90%, about 10% to about 80%, about 5% to about 70%, about 5% to about 60%, about 10% to about 50%, about 15% to about 40%, about 5% to about 20%, about 1% to about 20%, about 10% to about 30%, about 5%, about 10%, about 20%, about 30%, about 40%, about 50%, about 60%, about 70%, about 80%, about 90%, about 100%, about 10% to about 90%, about 12.5% to about 80%, about 20% to about 70%, about 25% to about 60%, or about 25% to about 50%, about 2 fold, about 3 fold, about 4 fold, about 5 fold, about 6 fold, about 7 fold, about 8 fold, about 9 fold, about 10 fold, at least 1.1 fold, at least 1.2 fold, at least 1.3 fold, at least 1.4 fold, at least 1.5 fold, at least 1.6 fold, at least 1.8 fold, at least 2 fold, at least 5 fold, at least 10 fold, at least 15 fold, at least 20 fold, at least 50 fold, at least 100 fold, at least 120 fold, from about 2 fold to about 500 fold, from about 1.1 fold to about 10 fold, from about 1.1 fold to about 5 fold, from about 1.5 fold to about 5 fold, from about 2 fold to about 5 fold, from about 3 fold to about 4 fold, from about 5 fold to about 10 fold, from about 5 fold to about 200 fold, from about 10 fold to about 150 fold, from about 10 fold to about 20 fold, from about 20 fold to about 150 fold, from about 20 fold to about 50 fold, from about 30 fold to about 150 fold, from about 50 fold to about 100 fold, from about 70 fold to about 150 fold, from about 100 fold to about 150 fold, from about 10 fold to about 100 fold, from about 100 fold to about 200 fold, compared to its (or their) level obtained in step (b), the therapy is considered to be effective. An effective therapy may be continued, or discontinued if the patient's condition has improved and is no longer in need of treatment. An ineffective treatment may be altered or modified, or replaced with other treatment.

The present methods can include the steps of measuring the level of at least one exosomal protein/polypeptide in a sample from a patient receiving a therapeutic intervention, and comparing the measured level to a reference level or the level of at least one exosomal protein/polypeptide in a control sample. The measured level of the at least one exosomal protein/polypeptide is indicative of the therapeutic efficacy of the therapeutic intervention.

Based on the measured exosomal protein/polypeptide levels, therapy may be continued or altered, e.g., by change of dose or dosing frequency, or by addition of other active agents, or change of therapeutic regimen altogether.

The present invention also encompasses a method of predicting or assessing the level of severity of transplant rejection in a patient. In one embodiment, the method comprises measuring the level of at least one exosomal protein/polypeptide in a biological sample from a patient, and comparing the measured level to a reference level or the level of the at least one exosomal protein/polypeptide in a control sample, wherein the measured level of the at least one exosomal protein/polypeptide is indicative of the level of severity of transplant rejection in the patient. In other embodiments, an increase or decrease (as described herein) in the level of the exosomal proteins/polypeptides is indicative of the level of severity of transplant rejection in the patient.

The expression profile of the exosomal proteins/polypeptides in a patient who has received a transplant may be determined/detected. The expression profile of the exosomal proteins/polypeptides of the patient may be compared with a reference value, where the reference value is based on a set of exosomal protein/polypeptide expression profiles of a transplant recipient without transplant rejection, and/or based on a set of exosomal protein/polypeptide expression profiles in an unaffected individual or unaffected individuals, and/or based on a set of exosomal protein/polypeptide expression profiles in the patient before, after and/or during therapy. The changes in exosomal protein/polypeptide expression may be used to alter or direct therapy, including, but not limited to, initiating, altering or stopping therapy.

Another aspect of the disclosure is a kit containing a reagent for measuring at least one exosomal protein/polypeptide in a biological sample, instructions for measuring at least one exosomal protein/polypeptide, and instructions for evaluating or monitoring transplant rejection in a patient based on the level of the at least one exosomal protein/polypeptide. In some embodiments, the kit contains reagents for measuring from 1 to about 20 human exosomal proteins/polypeptides, including 1, 2, 3, 4, 5, 6, 7, 8, 9, 10, 11, 12, 13, 14, 15, 16, 17, 18, 19, 20 up to n exosomal proteins/polypeptides. Also encompassed by the disclosure are kits for assessing or predicting the severity or progression of transplant rejection in a subject. The kit may comprise a reagent for measuring at least one exosomal protein/polypeptide in a biological sample, and instructions for assessing severity or progression of transplant rejection based on the level of the at least one exosomal protein/polypeptide. The kit may comprise one or biochips to assay the levels of a plurality exosomal proteins/polypeptides.

### Exosomal Proteins/Polypeptides

The present application measures the level of at least one exosomal protein/polypeptide in a biological sample. Samples can include any biological sample from which exosomal proteins/polypeptides can be isolated.

In certain embodiments, the sample is a body fluid. For example, the body fluid can include, but are not limited to, serum, plasma, blood, whole blood and derivatives thereof, urine, tears, saliva, sweat, cerebrospinal fluid (CSF), oral mucus, vaginal mucus, seminal plasma, semen, prostatic fluid, excreta, ascites, lymph, bile, and amniotic fluid. In certain embodiments, the biological sample is plasma or serum.

In certain embodiment, samples can include, but are not limited to, cardiac tissue, bone marrow, myocardium, endothelium, skin, hair, hair follicles, epithelial tissues, as well as other samples or biopsies. In certain embodiments, the biological sample is cardiac tissue.

The sample may be obtained at any time point alter the transplant procedure, such as about 10 minutes, about 30 minutes, about 1 hour, about 2 hours, about 3 hours, about 4 hours, about 5 hours, about 6 hours, about 7 hours, about 8 hours, about 10 hours, about 12 hours, about 15 hours, about 18 hours, about 20 hours, about 22 hours, about 1 day, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, about 3 years, about 5 years or longer following the transplantation procedure. The time point may also be earlier or later.

Exosomes may be isolated from the sample. Exosomes are cell-derived vesicles that are present in many biological fluids. In certain embodiments, their size may range from about 30 nm to about 100 nm. Exosomes contain various molecular constituents of their cell of origin, including, but not limited to, proteins, RNA (such as mRNA, miRNA), lipids and DNA. In certain embodiments, exosomes remain intact in biofluids during long-term storage.

Exosome may be isolated by any suitable techniques, including ultracentrifugation, micro-filtration, size-exclusion chromatography etc. or a combination thereof. Exosome can be isolated using a combination of techniques based on both physical (e.g. size, density) and biochemical parameters (e.g. presence/absence of certain proteins involved in their biogenesis). In certain embodiments, exosomes are isolated using a kit. In one embodiment, exosomes are isolated from serum using the Total Exosome Isolation Kit and/or the Total Exosome Isolation Reagent from Invitrogen.

In certain embodiments, 1 or more, 2 or more, 3 or more, 4 or more, 5 or more, 10 or more, 15 or more, 20 or more, 25 or more, 30 or more, 35 or more, 40 or more, 45 or all, exosomal proteins/polypeptides selected from LV102, FIBG, FIBB, FIBA, ACTB, ECM1, F13A. C1R, FINC, TSP1, TNNC1, FSVV04, STK36, IGJ, TOP2A, LV101, TRIPB, GK, LICAM, PON1, C1RL, ITIH2, KLKB1, HV315, APOL1, GELS, IGHD, IT1H1, FRMPD1, PLMN, KV302, FSW6P5, C9JMH6, B7ZKJS, KV1A1, F5H7E1, A1AG1, A2AP, HV304, GSJLSS, E9PBC5, QSVY30, Q5T9S5, C9JA05, and F5H4W9, or selected from the exosomal polypeptides/proteins in Table 1, FIG. 1A, FIG. 2A, FIG. 3A, FIG. 4, FIG. 5, FIG. 6B, FIG. 6C, FIG. 7, FIG. 8, and combinations thereof, are measured. In some embodiments, a panel of no greater than 20, no greater than 15, no greater than 10, or no greater than 5 exosomal proteins/polypeptides is tested, the panel including 1, 2, 3, 4, 5, 6, 7, 8, 9, 10, 11, 12, 13, 14, 15, 16, 17, 18, 19, 20 or more exosomal proteins/polypeptides as described herein.

The level or amount of exosomal protein/polypeptide in a patient sample can be compared to a reference level or amount of the exosomal protein/polypeptide present in a control sample. The control sample may be from a patient who has received a transplant without rejection or a plurality of patients who have received a transplant without rejection. The control sample may be from a healthy subject or a plurality of healthy subjects. In other embodiments, a control sample is taken from a patient prior to treatment with a therapeutic intervention or a sample taken from an untreated patient (e.g., a patient who has not received a transplant and/or an immunosuppressant therapy). Reference levels for an exosomal protein/polypeptide can be determined by determining the level of an exosomal protein/polypeptide in a sufficiently large number of samples obtained from a patient or patients who have received a transplant without transplant rejection, or normal, healthy control subjects to obtain a pre-determined reference or threshold value. A reference level can also be determined by determining the level of the exosomal protein/polypeptide in a sample from a patient prior to treatment with the therapeutic intervention.

Reference (or calibrator) level information and methods for determining reference levels can be obtained from publicly available databases, as well as other sources. (See, e.g., Bunk, D. M. (2007) Clin. Biochem. Rev., 28(4):131-137; and Remington: The Science and Practice of Pharmacy, Twenty First Edition (2005)).

### Protein-Based Assays

The level of an exosomal protein/polypeptide can be detected and/or quantified by any of a number of methods well known to those of skill in the art. The exosomal polypeptides/proteins may be detected by, for example, mass spectrometry (e.g., LC-MS/MS) and Western blot. The methods may include various immunoassays such as enzyme-linked immunosorbent assay (ELISA), lateral flow immunoassay (LFIA), immunohistochemistry, antibody sandwich capture assay, immunofluorescent assay, Western blot, enzyme-linked immunospot assay (EliSpot assay), precipitation reactions (in a fluid or gel), immunodiffusion, immunoelectrophoresis, radioimmunoassay (RIA), competitive binding protein assays, chemiluminescent assays, and the like. Also included are analytic biochemical methods such as electrophoresis, capillary electrophoresis, high-performance liquid chromatography (HPLC), thin layer chromatography (TLC), hyperdiffusion chromatography, liquid chromatography-tandem mass spectrometry, and the like. U.S. Pat. Nos. 4,366,241; 4,376,110; 4,517,288; and 4,837,168. Methods in Cell Biology Volume 37: Antibodies in Cell Biology, Asai, ed. Academic Press, Inc. New York (1993); Basic and Clinical Immunology 7th Edition. Stites & Tenrr, eds. (1991).

The level of an exosomal protein/polypeptide may be detected by using molecules (e.g., polypeptides, etc.) that bind to the exosomal protein/polypeptide. For example, the binding polypeptide may be an antibody or antibody fragment, such as an Fab, F(ab)₂, F(ab′)₂, Fd, or Fv fragment of an antibody. Any of the various types of antibodies can be used for this purpose, including, but not limited to, polyclonal antibodies, monoclonal antibodies, humanized antibodies, human antibodies (e.g., generated using transgenic mice, etc.), single chain antibodies (e.g., single chain Fv (scFv) antibodies), heavy chain antibodies and chimeric antibodies. The antibodies can be from various species, such as rabbits, mice, rats, goats, chickens, guinea pigs, hamsters, horses, sheep, llamas etc.

In certain embodiments, ELISA is used to detect and/or quantify one or more exosomal proteins/polypeptides in a sample. The ELISA can be any suitable methods, including, but not limited to, direct ELISA, sandwich ELISA, and competitive ELISA.

In certain embodiments, Western blot (immunoblot) is used to detect and quantify one or more exosomal proteins/polypeptides in a sample. The technique may comprise separating sample proteins by gel electrophoresis, transferring the separated proteins to a suitable solid support, and incubating the sample with the antibodies that specifically bind the one or more exosomal proteins/polypeptides.

The disclosure further includes protein microarrays (including antibody arrays) for the analysis of levels of a plurality of exosomal proteins/polypeptides. Protein microarray technology, which is also known as protein chip technology and solid-phase protein array technology, is well known to those of ordinary skill in the art. Protein microarray may be based on, but not limited to, obtaining an array of identified peptides or proteins on a fixed substrate, binding target molecules or biological constituents to the peptides, and evaluating such binding. See, e.g., MacBeath et al., Printing Proteins as Microarrays for High-Throughput Function Determination, Science 289(5485):1760-1763, 2000. In some embodiments, one or more control peptide or protein molecules are attached to the substrate.

The polypeptides that may be used to assay the level of an exosomal protein/polypeptide may be derived also from sources other than antibody technology. For example, such binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptides and non-peptide synthetic moieties. The exosomal protein/polypeptide can be used to screen peptide libraries, including phage display libraries, to identify and select peptide binding partners of the exosomal protein/polypeptide. Yeast two-hybrid screening methods also may be used to identify polypeptides that bind to the exosomal protein/polypeptide.

### Nucleic Acid-Based Assays

The present methods may also assay the presence of or quantity the gene encoding an exosomal protein/polypeptide or the gene product. Gene products include nucleic acids (e.g. mRNAs) derived from the gene.

The level of the DNA or RNA (e.g., mRNA) molecules may be determined/detected using routine methods known to those of ordinary skill in the art. The measurement result may be an absolute value or may be relative (e.g., relative to a reference oligonucleotide, relative to a reference mRNA, etc.). The level of the nucleic acid molecule may be determined/detected by nucleic acid hybridization using a nucleic acid probe, or by nucleic acid amplification using one or more nucleic acid primers.

Nucleic acid hybridization can be performed using Southern blots, Northern blots, nucleic acid microarrays, etc.

For example, the DNA encoding an exosomal protein/polypeptide in a sample may be evaluated by a Southern blot. Similarly, a Northern blot may be used to detect an exosomal protein/polypeptide mRNA. In one embodiment, mRNA is isolated from a given sample, and then electrophoresed to separate the mRNA species. The mRNA is transferred from the gel to a solid support. Labeled probes are used to identify or quantity the exosomal protein/polypeptide nucleic acids.

In certain embodiments, labeled nucleic acids are used to detect hybridization. Complementary nucleic acids may be labeled by any one of several methods typically used to detect the presence of hybridized polynucleotides. One method of detection is the use of autoradiography. Other labels include ligands that bind to labeled antibodies, fluorophores, chemiluminescent agents, enzymes, and antibodies which can serve as specific binding pair members for a labeled ligand.

Nucleic acid microarray technology, which is also known as DNA chip technology, gene chip technology, and solid-phase nucleic acid array technology, may be based on, but not limited to, obtaining an array of identified nucleic acid probes on a fixed substrate, labeling target molecules with reporter molecules (e.g., radioactive, chemiluminescent, or fluorescent tags such as fluorescein, Cye3-dUTP, or Cye5-dUTP, etc.), hybridizing target nucleic acids to the probes, and evaluating target-probe hybridization. Jackson et al. (1996) Nature Biotechnology, 14: 1685-1691. Chee et al. (1995) Science, 274: 610-613.

The sensitivity of the assays may be enhanced through use of a nucleic acid amplification system that multiplies the target nucleic acid being detected.

Nucleic acid amplification assays include, but are not limited to, the polymerase chain reaction (PCR), reverse transcription polymerase chain reaction (RT-PCR), real-time RT-PCR, quantitative RT-PCR, etc.

Measuring or detecting the amount or level of mRNA in a sample can be performed in any manner known to one skilled in the art and such techniques for measuring or detecting the level of an mRNA are well known and can be readily employed. A variety of methods for detecting mRNAs have been described and may include, Northern blotting, microarrays, real-time PCR, RT-PCR, targeted RT-PCR, in situ hybridization, deep-sequencing, single-molecule direct RNA sequencing (RNAseq), bioluminescent methods, bioluminescent protein reassembly, BRET (bioluminescence resonance energy transfer)-based methods, fluorescence correlation spectroscopy and surface-enhanced Raman spectroscopy (Cissell, K. A. and Deo, S. K. (2009) Anal. Bioanal. Chem., 394:1109-1116).

The methods of the present invention may include the step of reverse transcribing RNA when assaying the level or amount of an mRNA.

These assays of determining/detecting the presence and/or level of one or more exosomal proteins/polypeptides may include use of a label(s). The labels can be any material having a detectable physical or chemical property. Thus, a label is any composition detectable by spectroscopic, photochemical, biochemical, immunochemical, electrical, optical or chemical means. Such labels may include, but are not limited to, a fluorescent label, a radiolabel, a chemiluminescent label, an enzyme, a metallic label, a bioluminescent label, a chromophore, biotin etc. For example, a fluorescently labeled or radiolabeled antibody that selectively binds to a polypeptide of the invention may be contacted with a tissue or cell to visualize the polypeptide. In some aspects of the invention, a label may be a combination of the foregoing molecule types.

The level, amount, abundance or concentration of one or more exosomal proteins/polypeptides may be measured. The measurement result may be an absolute value or may be relative (e.g., relative to a reference protein or polypeptide, etc.)

In one embodiment, a difference (increase or decrease) in the measured level of the exosomal protein/polypeptide relative to the level of the exosomal protein/polypeptide in the control sample (e.g., a sample in at least one patient who has received a transplant without rejection, in the patient prior to treatment, at a different time point during treatment, or an untreated patient) or a pre-determined reference value is indicative of the therapeutic efficacy of the therapeutic intervention (e.g., an immunosuppressant therapy). In another embodiment, an increase (or decrease) in the measured level of the exosomal protein/polypeptide relative to the level of the exosomal protein/polypeptide in the control sample or pre-determined reference value is indicative of the therapeutic efficacy of the therapeutic intervention. For instance, in such embodiments, when the level of one or more exosomal proteins/polypeptides is increased (or decreased) when compared to the level in a control sample or pre-determined reference value in response to a therapeutic intervention, the increase (or decrease) is indicative of therapeutic efficacy of the therapeutic intervention.

In certain embodiments, a reduction or decrease in the measured level of the exosomal protein/polypeptide relative to the level of the exosomal protein/polypeptide in the control sample (e.g., a sample in the patient prior to treatment or an untreated patient) or pre-determined reference value can be indicative of the therapeutic efficacy of the therapeutic intervention. For instance, in such embodiments, when the level of one or more exosomal proteins/polypeptides is decreased (or increased) when compared to the level in a control sample or pre-determined reference value in response to a therapeutic intervention, the decrease (or increase) is indicative of therapeutic efficacy of the therapeutic intervention.

Patients showing different (elevated or reduced) levels of one or more exosomal proteins/polypeptides can be identified. The expression profile of these exosomal proteins/polypeptides may be used to calculate a score for the combined or individual exosomal protein/polypeptide expression. The scores of these patients will be compared to the score of unaffected individuals (e.g., patients without transplant rejection). The clinical condition of these patients with respect to their cardiac status may be correlated with the exosomal protein/polypeptide expression profiles. The scores may be used to identify groups of patients having transplant rejection responsive to immunosuppressant treatment.

### Transplant Rejection

The present method may be used to assess the transplant status or outcome, including, but not limited to, transplant rejection, transplant function (including delayed graft function), non-rejection based allograft injury, transplant survival, chronic transplant injury, or titer pharmacological immunosuppression. In some embodiments, the non-rejection based allograft injury may include ischemic injury, virus infection, peri-operative ischemia, reperfusion injury, hypertension, physiological stress, injuries due to reactive oxygen species and/or injuries caused by pharmaceutical agents. The transplant status or outcome may comprise vascular complications or neoplastic involvement of the transplanted organ.

In some embodiments, the methods described herein are used for diagnosing or predicting transplant status or outcome (e.g., transplant rejection). In some embodiments, the methods described herein are used to detect and/or quantify target exosomal proteins/polypeptides to determine whether a subject is undergoing transplant rejection. In some embodiments, the methods described herein are used to detect and/or quantify target exosomal proteins/polypeptides for diagnosis or prediction of transplant rejection. In some embodiments, the methods described herein are used to detect and/or quantify target exosomal proteins/polypeptides for determining an immunosuppressive regimen for a subject who has received a transplant. In some embodiments, the methods described herein are used to detect and/or quantify target exosomal proteins/polypeptides to predict transplant survival in a subject that have received a transplant. The invention provides methods of diagnosing or predicting whether a transplant in a transplant recipient will survive or be lost. In certain embodiments, the methods described herein are used to detect and/or quantify target exosomal proteins/polypeptides to diagnose or predict the presence of long-term graft survival. In some embodiments, the methods described herein are used to detect and/or quantify target exosomal proteins/polypeptides for diagnosis or prediction of non-rejection based transplant injury. The present methods may be used to diagnose graft-versus-host-disease (GVHD).

As used herein the term “diagnose” or “diagnosis” of a transplant status or outcome includes predicting or diagnosing the transplant status or outcome, determining predisposition to a transplant status or outcome, monitoring treatment of transplant patient, diagnosing a therapeutic response of transplant patient, and prognosis of transplant status or outcome, transplant progression, and response to a particular treatment.

The transplant may be an allograft or a xenograft. An allograft is a transplant of an organ, tissue, bodily fluid or cell from one individual to a genetically non-identical individual of the same species. A xenograft is a transplant of an organ, tissue, bodily fluid or cell from a different species.

The transplant maybe any organ or tissue transplant, including, but not limited to, a heart transplant, a kidney transplant, a liver transplant, a pancreas transplant, a lung transplant, an intestine transplant, a skin transplant, a bone marrow transplant, a small bowel transplant, a trachea transplant, a cornea transplant, a limb transplant, and a combination thereof.

The present methods may determine the presence, type and/or severity of the transplant rejection. Transplant rejection includes a partial or complete immune response to a transplanted cell, tissue, organ, or the like on or in a recipient of said transplant due to an immune response to a transplant. A transplant can be rejected through either a cell-mediated rejection (CMR) or antibody-mediated rejection (AMR). The rejection may be acute cellular rejection (ACR).

Rejection after a heart transplant may be graded according to the ISHLT (International Society for Heart and Lung Transplantation) guidelines (Table 2 and Table 3).

TABLE 2
ISHLT Standardized Cardiac Biopsy Grading (2004):
Acute Cellular Rejection (ACR)

| | |
|---|---|
| Grade 0R | No Rejection |
| Grade 1R, mild | Interstitial and/or perivascular infiltrate with |
| | up to 1 focus of myocyte damage |
| Grade 2R, moderate | Two or more foci of infiltrate with associated |
| | myocyte damage |
| Grade 3R, severe | Diffuse infiltrate with multifocal myocyte |
| | damage ± edema, ± hemorrhage ± vasculitis |

TABLE 3
ISHLT Recommendations for Acute Antibody-Mediated
Rejection (AMR) (2004)

| | |
|---|---|
| AMR 0 | Negative for acute antibody-mediated rejection |
| | No histologic or immunopathologic features of AMR |
| AMR 1 | Positive for AMR |
| | Histologic features of AMR |
| | Positive immunofluorescence or immunoperoxidase |
| | staining for AMR (positive CD68, C4d) |

The present methods may diagnose or predict any type of transplant rejection, including, but not limited to, hyperacute rejection, acute rejection, and/or chronic rejection. Hyperacute rejection can occur within minutes or hours to days following transplantation and may be mediated by a complement response in recipients with pre-existing antibodies to the donor. In hyperacute rejection, antibodies are observed in the transplant vasculature very soon after transplantation, possibly leading to clotting, ischemia, and eventual necrosis and death. Acute rejection occurs days to months or even years following transplantation. It can include a T-cell mediated response and is identified based on presence of T-cell infiltration of the transplanted tissue, structural injury to the transplanted tissue, and injury to the vasculature of the transplanted tissue. Chronic rejection occurs months to years following transplantation and is associated with chronic inflammatory and immune response against the transplanted tissue. Chronic rejection may also include chronic allograft vasculopathy, which is associated with fibrosis of vasculature of the transplanted tissue. U.S. Pat. No. 8,637,038. Fibrosis is a common factor in chronic rejection of all types of organ transplants. Chronic rejection can typically be described by a range of specific disorders that are characteristic of the particular organ. For example, in heart transplants or transplants of cardiac tissue, such as valve replacements, such disorders include fibrotic atherosclerosis; in lung transplants, such disorders include fibroproliferative destruction of the airway (bronchiolitis obliterans); in kidney transplants, such disorders include obstructive nephropathy, nephrosclerorsis, tubulointerstitial nephropathy; and in liver transplants, such disorders include disappearing bile duct syndrome. Chronic rejection can also be characterized by ischemic insult, denervation of the transplanted tissue, hyperlipidemia and hypertension associated with immunosuppressive drugs.

In some embodiments, the invention provides methods of determining whether a patient or subject is displaying transplant tolerance. The term “transplant tolerance” includes when the subject does not reject a graft organ, tissue or cell(s) that has been introduced into/onto the subject. In other words, the subject tolerates or maintains the organ, tissue or cell(s) that has been transplanted.

Graft-versus-host-disease (GVHD) is the pathological reaction that occurs between the host and grafted tissue. The grafted or donor tissue dominates the pathological reaction. GVHD can be seen following stem cell and/or solid organ transplantation. GVHD occurs in immunocompromised subjects, who when transplanted, receive “passenger” lymphocytes in the transplanted stem cells or solid organ. These lymphocytes recognize the recipient's tissue as foreign. Thus, they attack and mount an inflammatory and destructive response in the recipient. GVHD has a predilection for epithelial tissues, especially skin, liver, and mucosa of the gastrointestinal tract. GVHD subjects are immunocompromised due the fact that prior to transplant of the graft, the subject receives immunosuppressive therapy.

Certain embodiments of the invention provide methods of predicting transplant survival in a subject that has received a transplant. The invention provides methods of diagnosing or predicting whether a transplant in a transplant patient or subject will survive or be lost. In certain embodiments, the invention provides methods of diagnosing or predicting the presence of long-term graft survival. Long-term graft survival refers to graft survival for at least about 5 years beyond current sampling, despite the occurrence of one or more prior episodes of acute rejection. In certain embodiments, transplant survival is determined for patients in which at least one episode of acute rejection has occurred. As such, these embodiments provide methods of determining or predicting transplant survival following acute rejection.

The level of one or more exosomal proteins/polypeptides may be assayed to diagnose or monitor other cardiac disease states including, but not limited to, diseases of the cardiac valves, other forms of cardiomyopathies, inflammatory heart disease, congenital heart disease.

### Therapeutic Intervention

Based on the levels of the exosomal protein(s)/polypeptide(s), transplant rejection may be diagnosed or predicted (a risk of transplant rejection assessed), and then the subject may be treated with a therapy for the rejection, such as an immunosuppressant therapy.

An immunosuppressant, also referred to as an immunosuppressive agent, can be any compound that decreases the function or activity of one or more aspects of the immune system, such as a component of the humoral or cellular immune system or the complement system.

Non-limiting examples of immunosuppressants include, (1) antimetabolites, such as purine synthesis inhibitors (such as inosine monophosphate dehydrogenase (IMPDH) inhibitors, e.g., azathioprine, mycophenolate, and mycophenolate mofetil), pyrimidine synthesis inhibitors (e.g., leflunomide and teriflunomide), and antifolates (e.g., methotrexate); (2) calcineurin inhibitors, such as tacrolimus, cyclosporine A, pimecrolimus, and voclosporin; (3) TNF-alpha inhibitors, such as thalidomide and lenalidomide; (4) IL-1 receptor antagonists, such as anakinra; (5) mammalian target of rapamycin (mTOR) inhibitors, such as rapamycin (sirolimus), deforolimus, everolimus, temsirolimus, zotarolimus, and biolimus A9; (6) corticosteroids, such as prednisone; and (7) antibodies to any one of a number of cellular or serum targets (including anti-lymphocyte globulin and anti-thymocyte globulin).

Non-limiting exemplary cellular targets and their respective inhibitor compounds include, but are not limited to, complement component 5 (e.g., eculizumab); tumor necrosis factors (TNFs) (e.g., infliximab, adalimumab, certolizumab pegol, afelimomab and golimumab); IL-5 (e.g., mepolizumab); IgE (e.g., omalizumab); BAYX (e.g., nerelimomab); interferon (e.g., faralimomab); IL-6 (e.g., elsilimomab); IL-12 and IL-13 (e.g., lebrikizumab and ustekinumab); CD3 (e.g., muromonab-CD3, otelixizumab, teplizumab, visilizumab); CD4 (e.g., clenoliximab, keliximab and ranolimumab); CD11a (e.g., efalizumab); CD18 (e.g., erlizumab); CD20 (e.g., afutuzumab, ocrelizumab, pascolizumab); CD23 (e.g., lumiliximab); CD40 (e.g., teneliximab, toralizumab); CD62L/L-selectin (e.g., aselizumab); CD80 (e.g., galiximab); CD147/basigin (e.g., gavilimomab); CD154 (e.g., ruplizumab); BLyS (e.g., belimumab); CTLA-4 (e.g., ipilimumab, tremelimumab); CAT (e.g., bertilimumab, lerdelimumab, metelimumab); integrin (e.g., natalizumab); IL-6 receptor (e.g., tocilizumab); LFA-1 (e.g., odulimomab); and IL-2 receptor/CD25 (e.g., basiliximab, daclizmnab, inolimomab).

The present disclosure provides for methods of evaluating and/or monitoring the efficacy of a therapeutic intervention (e.g., an immunosuppressant therapy) for treating transplant rejection. These methods can include the step of measuring the level of at least one exosomal protein/polypeptide, or a panel of exosomal proteins/polypeptides, in a biological sample from a patient who has received a transplant. In some embodiments, the level of the at least one exosomal protein/polypeptide in the biological sample is compared to a reference level, or the level of the at least one exosomal protein/polypeptide in a control sample. The control sample may be taken from the patient at a different time point after transplantation, or from the patient before initiation of the therapeutic intervention (e.g., an immunosuppressant therapy), or from the patient at a different time point after initiation of the therapeutic intervention (e.g., an immunosuppressant therapy). The measured level of the at least one exosomal protein/polypeptide is indicative of the therapeutic efficacy of the therapeutic intervention. In some cases, an increase or decrease in the level of the exosomal protein/polypeptide is indicative of the efficacy of the therapeutic intervention. In some embodiments, a change in the measured level of the at least one exosomal protein/polypeptide relative to a sample from the patient taken prior to treatment or earlier during the treatment regimen is indicative of the therapeutic efficacy of the therapeutic intervention.

In certain embodiments, the method comprises detecting 1, 2, 3, 4, 5, 6, 7, 8, 9, 10, 11, 12, 13, 14, 15, 16, 17, 18, 19, 20 or more exosomal proteins/polypeptides described herein. When the levels of a panel of exosomal proteins/polypeptides are determined/detected in the patient sample, the patient sample may be classified as indicative of effective or non-effective intervention on the basis of a classifier algorithm. For example, samples may be classified on the basis of threshold values as described, or based upon mean and/or median exosomal protein/polypeptide levels in one population or versus another (e.g., a population of healthy controls or a population of patients having received a transplant without rejection, or levels based on effective versus ineffective therapy).

Various classification schemes are known for classifying samples between two or more classes or groups, and these include, without limitation: Principal Components Analysis, Naive Bayes, Support Vector Machines, Nearest Neighbors, Decision Trees, Logistic, Artificial Neural Networks, Penalized Logistic Regression, and Rule-based schemes. In addition, the predictions from multiple models can be combined to generate an overall prediction. Thus, a classification algorithm or “class predictor” may be constructed to classify samples. The process for preparing a suitable class predictor (reviewed in Simon (2003) British Journal of Cancer (89) 1599-1604).

The present invention also provides methods for modifying a treatment regimen comprising detecting the level of at least one exosomal protein/polypeptide in a biological sample from a patient receiving the therapeutic intervention and modifying the treatment regimen based on an increase or decrease in the level of the at least one exosomal protein/polypeptide in the biological sample. The methods for modifying the treatment regimen of a therapeutic intervention may comprise the steps of: (a) detecting the level of at least one exosomal protein/polypeptide in a biological sample from a patient receiving the therapeutic intervention; and (b) modifying the treatment regimen based on an increase or decrease in the level of the at least one exosomal protein/polypeptide in the biological sample. In some embodiments, the method comprises detecting 2, 3, 4, 5, 6, 7, 8, 9, 10 or more exosomal proteins/polypeptides described herein. In certain embodiments, the levels of less than 50, less than 30, or less than 20 exosomal proteins/polypeptides are detected.

Modifying the treatment regimen can include, but is not limited to, changing and/or modifying the type of therapeutic intervention, the dosage at which the therapeutic intervention is administered, the frequency of administration of the therapeutic intervention, the route of administration of the therapeutic intervention, as well as any other parameters that would be well known by a physician to change and/or modify. For example, where one or more exosomal proteins/polypeptides decrease (or increase) during therapy or match reference levels, the therapeutic intervention is continued. In embodiments where one or more exosomal proteins/polypeptides do not decrease (or increase) during therapy or match reference levels, the therapeutic intervention is modified. In another embodiment, the information regarding the increase or decrease in the level of at least one exosomal protein/polypeptide can be used to determine the treatment efficacy, as well as to tailor the treatment regimens of therapeutic interventions.

In one embodiment, the present methods are used for the titration of a subject's immunosuppression. Additionally, the present method can be utilized to determine whether the response to drug therapy indicates resolution of rejection risk. It can also be used to test whether the reduction of drug therapy increases the risk of rejection and whether drug therapy, if discontinued, should be resumed. This helps avoiding over-medication and/or under-medication of a given patient and duration of treatment can be tailored to the needs of the patient. The titration of immunosuppression can be after organ transplantation, or during a viral or bacterial infection. Further, the titration can be during a viral or bacterial infection after a subject has undergone organ transplantation. The method can include monitoring the response of a subject to one or more immunosuppressive agents, the withdrawal of an immunosuppressive agent, an antiviral agent, or an anti-bacterial agent.

Information gained by the methods described herein can be used to develop a personalized treatment plan for a transplant recipient. Accordingly, the disclosure further provides methods for developing personalized treatment plans for transplant recipients. The methods can be carried out by, for example, carrying out any of the methods of exosomal protein/polypeptide analysis described herein and, in consideration of the results obtained, designing a treatment plan for the patient whose transplant is assessed. If the levels of exosomal proteins/polypeptides indicate that the patient is at risk for an undesirable clinical outcome (e.g., transplant rejection, developing delayed graft function, or compromised graft function), the patient is a candidate for treatment with an effective amount of an immunosuppressant. Depending on the level of exosomal proteins/polypeptides, the patient may require a treatment regime that is more aggressive than a standard regime, or it may be determined that the patient is best suited for a standard regime. When so treated, one can treat or prevent transplant rejection (or, at least, prolong the time the transplanted organ functions adequately). Conversely, a different result (i.e., a different level of exosomal proteins/polypeptides) may indicate that the patient is not likely to experience an undesirable clinical outcome. In that event, the patient may avoid immunosuppressants. U.S. Pat. No. 8,741,557.

### Samples

Sampling methods are well known by those skilled in the art and any applicable techniques for obtaining biological samples of any type are contemplated and can be employed with the methods of the present invention. (See, e.g., Clinical Proteomics: Methods and Protocols, Vol. 428 in Methods in Molecular Biology, Ed. Antonia Vlahou (2008).)

The samples may be drawn before, during or after transplantation. The samples may be drawn at different time points during transplantation, and/or be drawn at different time points after transplantation.

When the sample is drawn after transplantation, it can be obtained from the subject at any point following transplantation. In some embodiments, the sample is obtained about 1 week, about 2 weeks, about 3 weeks, about 1 month, about 2 months, about 3 months, about 4 months, about 5 months, about 6 months, at least 1, 2, 3, or 6 months following transplantation. In some embodiments, the sample is obtained least 1, 2, 3, 4, 6 or 8 weeks following transplantation. In some embodiments, the sample is obtained at least 1, 2, 3, 4, 5, 6, or 7 days following transplantation. In some embodiments, the sample is obtained at least 10 minutes, 30 minutes, 1 hour, 6 hours, 12 hours, 18 hours or 24 hours after transplantation. In other embodiments, the sample is obtained at least one week following transplantation. In some embodiments, one or more exosomal proteins/polypeptides are measured between 1 and 8 weeks, between 2 and 7 weeks, at 1, 2, 3, 4, 5, 6, 7 or 8 weeks following transplantation.

### Kits

Another aspect of the disclosure is a kit containing a reagent or reagents for measuring at least one exosomal protein/polypeptide in a biological sample, instructions for measuring the at least one exosomal protein/polypeptide, and/or instructions for evaluating or monitoring transplant rejection in a patient based on the level of the at least one exosomal protein/polypeptide, and/or instructions for assessing an immunosuppressant therapy in a patient. In some embodiments, the kit contains reagents for measuring from 1 to about 20 human exosomal proteins/polypeptides, including at least 1, 2, 3, 4, 5, 6, 7, 8, 9, 10, 11, 12, 13, 14, 15, 16, 17, 18, 19, 20 or more exosomal proteins/polypeptides as described herein.

In certain embodiments, the kit comprises antibodies specific to one or more exosomal proteins/polypeptides.

In certain embodiments, the kit comprises primers and/or probe for reverse transcribing, amplifying, and/or hybridizing to one or more mRNAs of one or more exosomal proteins/polypeptides. Such kits can further comprise one or more normalization controls and/or a TaqMan probe specific for each mRNA.

The invention may also encompass biochips. Biochips contain a microarray of molecules (e.g., antibodies, peptides etc. as described herein) which are capable of binding to the exosomal proteins/polypeptides described herein.

Any of the compositions described herein may be comprised in a kit. In one embodiment, the kit contains a reagent for measuring at least one exosomal protein/polypeptide in a biological sample, instructions for measuring the at least one exosomal protein/polypeptide, and instructions for evaluating or monitoring transplant rejection in a patient based on the level of the at least one exosomal protein/polypeptide. In some embodiments, the kit contains reagents for measuring the level of at least 2, 3, 4, 5, 6 or 10 (or more) exosomal proteins/polypeptides. The kit may also be customized for determining the efficacy of therapy for transplant rejection, and thus provides the reagents for determining 50 or fewer, 40 or fewer, 30 or fewer, or 25 or fewer exosomal proteins/polypeptides.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed (e.g., sterile, pharmaceutically acceptable buffer and/or other diluents). However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

When the components of the kit are provided in one and/or more liquid solutions, the liquid solution may be an aqueous solution. The components of the kit may also be provided as dried powder(s). When reagents and/or components are provided as a dry powder, the powder can be reconstituted by the addition of a suitable solvent. It is envisioned that the solvent may also be provided in another container means.

Such kits may also include components that preserve or maintain the reagents or that protect against their degradation. Such components may be protease inhibitors or protect against proteases. Such kits generally will comprise, in suitable means, distinct containers for each individual reagent or solution.

A kit will also include instructions for employing the kit components as well the use of any other reagent not included in the kit. Instructions may include variations that can be implemented.

The following are examples of the present invention and are not to be construed as limiting.

### Example 1

Exosomes are cell-derived circulating vesicles that play an important role in cell-cell communication. Exosomes are actively assembled and carry mRNAs, miRNAs and proteins. The gold standard for cardiac allograft surveillance is endomyocardial biopsy (EMB), an invasive technique with distinct complication profile. The development of novel, noninvasive methods for the early diagnosis of allograft rejection is warranted.

We hypothesized that the exosomal proteome is altered in rejection, allowing a distinction between non-rejection and rejection episodes.

Serum samples were collected from heart transplant (HTx) recipients with no rejection, acute cellular rejection and antibody-mediated rejection. LC-MS/MS analysis of serum exosome was performed using an Orbitrap Fusion Tribrid Mass Spectrometer.

Principal component analysis (PCA) revealed a clustering of 3 groups: (1) control and HF; (2) HTx and no rejection; and (3) ACR and AMR. A total of 45 proteins were identified that could distinguish between groups (q<0.05). Comparison of serum exosomal proteins from control, HF and non-rejection HTx revealed 17 differentially expressed proteins in at least one group (q<0.05). Finally, comparisons of non-rejection HTx, ACR and AMR serum exosomes revealed 15 differentially expressed proteins in at least one group (q<0.05). Of these 15 proteins, eight proteins are known to play a role in immune response.

Characterizing of circulating exosomal proteome in different cardiac disease states reveals unique protein expression patterns indicative of the respective pathologies. Our data suggest that HTx and allograft rejection alter the circulating exosomal protein content. Exosomal protein analysis could be a novel approach to detect and monitor transplant rejection and lead to the development of predictive and prognostic biomarkers.

### Material and Methods

### Patient Enrollment and Baseline Demographics

Study participants were divided into 5 groups: healthy controls (n=10); HF patients without allograft (n=10); HTx patients without rejection (n=10); and HTx patients undergoing ACR (n=10) or AMR (n=8). Control serum samples were collected from organ donors whose hearts were explanted but were not used for HTx. Non-allograft HF patients were recruited during visits at the outpatient HF Clinic at New York-Presbyterian Hospital/Columbia University Medical Center (NYP/CUMC). HTx patients were recruited following transition to a Step Down Unit after receiving their allograft. ACR or AMR cases among our study participants were identified based on EMB histopathology reports. All patients gave written informed consent to participate in the study, which was conducted in accordance with the protocol approved by the CUMC Institutional Review Board.

### Exosome Isolation

Exosomes were isolated from 200 μl of patient serum using a commercially available isolation kit (invitrogen, Total Exosome Isolation from Serum) according to manufacturer's instructions. This kit offers a poly-ethylene-glycol based method. Kit-based isolation methods have been shown to give exosome yield and purity comparable to the ultracentrifugation method.^{24,25} Total exosome lysate was then generated in 50 μl of the lysis buffer (50 mM Ammonium Bicarbonate, 4M Urea, and protease cocktail) using 1.4 mm ceramic beads and the Omni Bead Rupture Homogenizer (Omni International, GA). Protein concentration in total exosome lysate was determined by the EZQ Protein Quantification Assay (Life Technology Corp. CT).

### Mass Spectrometry

2 pig of exosome lysate from each patient were digested by trypsin and analyzed by liquid chromatography-tandem mass spectrometry (LC-MS/MS) by the Proteomics Shared Resource at the Herbert Irving Comprehensive Cancer Center of Columbia University Medical Center. LC-MS/MS was performed using an Orbitrap Fusion Tribrid Mass Spectrometer (Thermo). MS/MS spectra were cross-referenced against a human protein database obtained from UniProt (www.unipot.org, released in 5/2015) using the Proteome Discoverer software 1.4 (Thermo). Spectral counts (number of MS/MS) were used for relative quantification. Because duplicate accession numbers in the raw MS/MS data represented isoforms of a single full-length protein, their counts were included under their respective full-length sequences.

The concentrated peptide mix was reconstituted in a solution of 2% acetonitrile (ACN), 2% formic acid (FA) for MS analysis. Peptides were loaded with the autosampler directly onto a 2 cm C18 PepMap pre-column and were eluted from the 15 cm×75 μm ID PepMap RSLC C18, 3 μm column with a 70 min gradient from 2% buffer B to 30% buffer B (100% acetonitrile, 0.1% formic acid). The gradient was switched from 30% to 85% buffer B over 5 min and held constant for 5 min. Finally, the gradient was changed from 85% buffer B to 98% buffer A (100% water, 0.1% formic acid) over 1 min, and then held constant at 98% buffer A for 8 more minutes. The application of a 2.0 kV distal voltage electrosprayed the eluting peptides directly into the Orbitrap Fusion™ Tribrid mass spectrometer equipped with an Easy-spray source (Thermo Finnigan, San Jose, Calif.). Full mass spectra was recorded on the peptides over a 400 to 1500 m/z range at 120,000 resolution, followed by tandem mass (MS/MS) CID (collision induced dissociation) events for a total of a 3-sec cycle. Charge state dependent screening was turned off, and peptides with a charge state of 2-6 were analyzed. Mass spectrometer-scanning functions and HPLC gradients were controlled by the Xcalibur data system (Thermo Finnigan, San Jose, Calif.). Three technical replicates were run for each sample, and MS/MS data from technical replicates were merged for subsequent database search.

### Database Search and Interpretation of MS/MS Data

Tandem mass spectra from raw files were searched against a human protein database using the Proteome Discoverer (Thermo Finnigan, San Jose, Calif.). The Proteome Discoverer application extracts relevant MS/MS spectra from the .raw file and determines the precursor charge state and the quality of the fragmentation spectrum. The Proteome Discoverer probability-based scoring system rates the relevance of the best matches found by the SEQUEST algorithm.²⁶ The human database was downloaded as FASTA-formatted sequences from Uniprot protein database (database released in 0512015).²⁷ The peptide mass search tolerance was set to 10 ppm. A minimum sequence length of 7 amino acids residues was required. Only fully tryptic peptides were considered. To calculate confidence levels and false discovery rates (FDR), Proteome Discoverer generates a decoy database containing reverse sequences of the non-decoy protein database and performs the search against this concatenated database (non-decoy+decoy).²⁵ The discriminant score was set at 1% FDR determined based on the number of accepted decoy database peptides to generate protein lists for this study. Spectral counts were used as the quantitative values for the protein-based list (distinct proteins).

### Statistical Analysis

Principal component analysis (PCA), limma empirical Bayes analysis, and 2-group t-test of semi-quantitative MS data were performed as indicated using the Omics Explorer software (Qlucore). Adjusted p values or respectively q<0.05 was considered significant. Spectral counts are given as spectral counts±SD.

### Results

### Exosomal Protein Profiling Distinguishes Between Various Cardiac Pathologies

A total of 3537 proteins were identified based on a 1% false discovery rate (FDR) at the peptide level. Limma empirical Bayes analysis was applied to the semi-quantitative values (spectral counts) of the entire data, and differentially expressed protein were identified with a FDR threshold at 5% (q<0.05). Principal component analysis (PCA) applied to the data set identified an exosomal protein signature which distinguishes the following three patient groups: (1) control and HF; (2) HTx, no rejection; and (3) ACR and AMR. A total of 45 proteins were identified that could distinguish at least one group from the rest of the dataset at q<0.05 (FIG. 1).

Heart Failure and Heart Transplantation Status are Associated with Distinct Profiles in Exosomal Proteins Relative to Controls

Limma empirical Bayes analysis was applied to serum exosomal protein MS/MS data from the control, HF and non-rejection HTx cohorts and filtered at q<0.05. PCA gave distinct groupings for the data from each cohort. Expression of 17 proteins collectively were found to distinguish at least one group at 5% FDR (q<0.05) (FIG. 2). Of these 17 proteins, ten proteins play a role in inflammation and immunity. Six proteins have immunoglobulin (Ig) structural components: J chain (IGJ: control 24.70±6.41; HF 0.10±0.32; HTx 0.00±0.00; q<0.0001); Ig K chain V-III region SIE (KV302: control 45.30±12.93; HF 56.20±24.40; HTx 0.00±0.00; q<0.0001): Ig K chain V-III region NG9 (KV303: control 11.40; 4.81; HF 8.70±3.95; HTx 2.50±3.98; q=0.0125); Ig λ chain V-I region S107A (KV1A1: control 0.00±0.00; HF 5.40-3.66; HTx 0.00±0.00; q<0.0001); Ig λ chain V-I region VOR (LV101: control 3.30±3.09; HF 0.00±0.00; HTx 0.00) 0.00; q=0.0001); and Ig λ chain V-I region HA (LV102: control 0.00±0.00; IP 0.00±0.00; HTx 3.60±3.57; q=0.0341). Complement component 5 (CO5: control 212.90±39.58; HF 212.30±39.04; HTx 299.20±41.31; q=0.0119) and complement C1r subcomponent-like protein (C1RL: control 11.30±8.07; HF 8.60±8.51; HTx 0.00±0.00; q=0.0128) were also significantly different across these three cohorts. Two additional immune modulators were also identified: inter-alpha-trypsin inhibitor heavy chain H2 (ITIH2: control 143.60±29.52; HF 142.40±59.22; HTx 0.00±0.00; q<0.0001) and paraoxonase-1 (PON1: control 18.20±11.91; HF 21.40±19.01; HTx 0.001±0.00; q=0.0002).

In addition to the immune response, we found four proteins known to be hematological regulators: alpha-2-antiplasmin (A2AP: control 4.40±2.80; HF 0.00±0.00; HTx 4.00±2.87; q=0.0160); plasminogen (PLMN: control 81.30±7.92; HF 76.10±12.24; HTx 54.80±12.15; q=0.0034); fibrinogen beta chain (FIBB: control 3.1±4.012; HF 3.5±6.04; HTx 47.6±39.94; q=0.0067); and fibrinogen gamma chain (FIBG: control 3.00±3.23; HF 3.30±5.33; HTx 37.60±33.38; q=0.0115).

Two additional proteins varied significantly across groups: serine/threonine-protein kinase 36 (STK36) which has been lined to cell proliferation and homeostasis (control 5.00±2.98; HF 0.90±1.91; HTx 0.90±1.37; q=0.0128) and neural cell adhesion molecule L1 (LICAM) which is associated with cell migration (control 2.70±1.64; HF 0.30±0.48; HTx 0.60±0.84; q=0.0397) (FIG. 2).

Table 4 lists the levels of a number of exosomal proteins for control samples, HF samples and HTx samples.

TABLE 4

| | Control | HF | HTx |
|---|---|---|---|
| | (spectral | (spectral | (spectral |
| Protein | counts) | counts) | counts) |
| IGJ | 24.70 ± 6.41 | 0.10 ± 0.32 | 0.00 ± 0.00 |
| KV302 | 45.30 ± 12.93 | 56.20 ± 24.40 | 0.00 ± 0.00 |
| KV303 | 11.40 ± 4.81 | 8.70 ± 3.95 | 2.50 ± 3.98 |
| KV1A1 | 0.00 ± 0.00 | 5.40 ± 3.66 | 0.00 ± 0.00 |
| LV101 | 3.30 ± 3.09 | 0.00 ± 0.00 | 0.00 ± 0.00 |
| LV102 | 0.00 ± 0.00 | 0.00 ± 0.00 | 3.60 ± 3.57 |
| CO5 | 212.90 ± 39.58 | 212.30 ± 39.04 | 299.20 ± 41.31 |
| C1RL | 11.30 ± 8.07 | 8.60 ± 8.51 | 0.00 ± 0.00 |
| ITIH2 | 143.60 ± 29.52 | 142.40 ± 59.22 | 0.00 ± 0.00 |
| PON1 | 18.20 ± 11.91 | 21.40 ± 19.01 | 0.00 ± 0.00 |
| A2AP | 4.40 ± 2.80 | 0.00 ± 0.00 | 4.00 ± 2.87 |
| PLMN | 81.30 ± 7.92 | 76.10 ± 12.24 | 54.80 ± 12.15 |
| FIBB | 3.1 ± 4.012 | 3.5 ± 6.04 | 47.6 ± 39.94 |
| FIBG | 3.00 ± 3.23 | 3.30 ± 5.33 | 37.60 ± 33.38 |
| STK36 | 5.00 ± 2.98 | 0.90 ± 1.91 | 0.90 ± 1.37 |
| L1CAM | 2.70 ± 1.64 | 0.30 ± 0.48 | 0.60 ± 0.84 |

Allograft Rejection is Associated with Significant Changes in Exosomal Signatures of Immunological and Hematological Proteins when Compared to the Non-Rejection Profile

Limma empirical Bayes analysis of serum exosomal protein counts in non-rejection HTx, ACR and AMR samples identified 15 proteins that could distinguish at least one group from the dataset at q<0.05 (FIG. 3). PCA showed each of the 3 cohorts forming a distinct data cluster. Of these 15 proteins, 8 participate in the immune response. Two complement factor components were identified: complement C1q subcomponent subunit A (C1QA: HTx 130.60±52.59; ACR 64.90±15.65; AMR 57.75±20.77; q=0.0150); C1r subcomponent (C1R: HTx 120.10±41.62; ACR 60.30±18.34; AMR 55.50±13.67; q=0.0079). Three more proteins were Ig subfractions: KV302 (HTx 0.00±0.00; ACR 60.50±24.54; AMR 60.50±22.96; q<0.0001) and Ig heavy chain V-Ill regions TIL (HV304: HTx 0.00±0.00; ACR 15.00±8.26; AMR 13.88±9.31; q<0.0001) and WAS (HV315: HTx 13.70±4.64; ACR 0.00±0.00; AMR 0.00-0.00; q<0.0001). IT1H1 (HTx 76.00±16.26; ACR 99.00±23.03; AMR 0.00±0.00; q<0.0001) and apolipoprotein L1 (APOL1: HTx 25.70±10.30; ACR 0.00±0.00; AMR 0.00±0.00; q<0.0001) were also identified.

A total of 6 hematological proteins were also significantly different across cohorts: coagulation factor XIII A chain (F13A: HTx 26.10±23.23; ACR 0.80±1.14; AMR 0.38±1.06; q=0.0150); fibrinogen alpha chain (FIBA: HTx 56.40±36.93; ACR 15.70±8.00; AMR 10.00±7.31; q=0.0058); FIBB (HTx 47.60±39.94; ACR 5.00±8.27; AMR 1.63±3.85; q=0.0051); FIBG (HTx 37.60±33.38; ACR 3.10±5.63; AMR 0.75±1.49; q=0.0014); fibronectin (FINC: HTx 974.50±226.05; ACR 560.90±151.49; AMR 477.38±133.86; q=0.0009); and thrombospondin-1 (TSP1: HTx 148.70±69.99; ACR 60.30±23.67; AMR 42.00±18.97) (FIG. 3).

Also found to be significantly different across groups were FERM and PDZ domain-containing protein 1 (FRMPD1: HTx 0.00±0.00; ACR 3.10±2.28; AMR 0.00±0.00; q=0.0014) and β-actin (ACTB: HTx 4.20±2.53; ACR 0.00±0.00; AMR 0.00±0.00; q<0.0001).

Table 5 lists the levels of a number of exosomal proteins for HTx with no rejection samples, ACR samples and AMR samples, as well as level changes compared to HTx with no rejection.

TABLE 5

| | | ACR | | AMR | |
|---|---|---|---|---|---|
| | HTx (no | | Change | | Change |
| | rejection) | Level | Compared | Level | Compared |
| | (spectral | (spectral | to HTx (no | (spectral | to HTx (no |
| Protein | counts) | counts) | rejection) | counts) | rejection) |
| C1QA | 130.60 ± 52.59 | 64.90 ± 15.65 | Decrease~50.3% | 57.75 ± 20.77 | Decrease~55.8% |
| C1R | 120.10 ± 41.62 | 60.30 ± 18.34 | Decrease~49.8% | 55.50 ± 13.67 | Decrease~53.8% |
| KV302 | 0.00 ± 0.00 | 60.50 ± 24.54 | Increase | 60.50 ± 22.96 | Increase |
| HV304 | 0.00 ± 0.00 | 15.00 ± 8.26 | Increase | 13.88 ± 9.31 | Increase |
| HV315 | 13.70 ± 4.64 | 0.00 ± 0.00 | Decrease~100% | 0.00 ± 0.00 | Decrease~100% |
| ITIH1 | 76.00 ± 16.26 | 99.00 ± 23.03 | Increase~30.3% | 0.00 ± 0.00 | Decrease~100% |
| APOL1 | 25.70 ± 10.30 | 0.00 ± 0.00 | Decrease~100% | 0.00 ± 0.00 | Decrease~100% |
| HBB | 0.00 ± 0.00 | 3.00 ± 2.75 | Increase | 0.00 ± 0.00 | — |
| F13A | 26.10 ± 23.23 | 0.80 ± 1.14 | Decrease~96.9% | 0.38 ± 1.06 | Decrease~98.5% |
| FIBA | 56.40 ± 36.93 | 15.70 ± 8.00 | Decrease~72.2% | 10.00 ± 7.31 | Decrease~82.3% |
| FIBB | 47.60 ± 39.94 | 5.00 ± 8.27 | Decrease~89.5% | 1.63 ± 3.85 | Decrease~96.6% |
| FIBG | 37.60 ± 33.38 | 3.10 ± 5.63 | Decrease~91.8% | 0.75 ± 1.49 | Decrease~98% |
| FINC | 974.50 ± 226.05 | 560.90 ± 151.49 | Decrease~76.8% | 477.38 ± 133.86 | Decrease~51% |
| TSP1 | 148.70 ± 69.99 | 60.30 ± 23.67 | Decrease~59.4% | 42.00 ± 18.97 | Decrease~71.8% |
| FRMPD1 | 0.00 ± 0.00 | 3.10 ± 2.28 | Increase | 0.00 ± 0.00 | — |
| ACTB | 4.20 ± 2.53 | 0.00 ± 0.00 | Decrease~100% | 0.00 ± 0.00 | Decrease~100% |

### Discussion

Exosomes are secretory vesicles that are now known to play an increasingly important role in intercellular signaling.^{17,19} Particular interest has been directed toward elucidating the role of exosomes in immunity. Exosomes have been shown to modulate antigen presentation, cytokine production and cell proliferation both in vitro and in vivo.²⁹⁻³². Our study found that cardiac allograft rejection is linked to significant changes of the serum exosomal proteome, especially in proteins controlling immunity and hemostasis, compared to HTx patients not experiencing rejection.

MS/MS analysis was performed using serum-derived exosomes isolated from healthy controls, HF patients, HTx recipients without rejection and HTx patients experiencing ACR or AMR. Principal component analysis revealed that our cohorts could be represented as 3 distinct groups based on their serum exosomal protein profiles: (1) controls and HF patients; (2) HTx patients without rejection; and (3) ACR and AMR. Interestingly, control and HF samples showed greater similarity than controls and HTx without rejection. Despite the goal of transplantation to correct the pathology of HF, it therefore appears that the introduction of the healthy yet foreign allograft causes more drastic changes in cell-cell signaling rather than a return to pre-HF exosomal protein signatures. The preponderance of immune-related proteins identified by comparing control, HF and HTx samples suggests that this may be due to increased immune surveillance of the allograft. Control and HF samples could only be distinguished by their exosomal protein profiles at a nonsignificant q-value of 0.1, further suggesting that, despite the severity of symptoms, HF does not cause changes in exosome cargo as pronounced as one might expect.

We found a 15-protein signature that distinguishes HTx (non-rejection), ACR and AMR cohorts. Of these 15 proteins, two proteins were components of the complement cascade: C1QA and C1R. Three proteins were Ig subfractions: KV302, HV304, HV315. Six proteins play a role in coagulation: FIBA, FIBB, FIBG. FINC, F13A and TSP1. We also identified APOL1, which is a member of the Bcl-2 family of apoptotic proteins; is inducible by IFN-r and TNF-α; and can induce autophagic cell death.³³⁻³⁵ These proteins are of great interest not only because of the highly significant differences between rejection and non-rejection but also because of their roles in immune processes and hemostasis. C1QA and C1R, in addition to C1QB and C1QC, combine to form the C1 complex, which binds the Fc region of antigen-bound IgG or IgM to initiate the classical complement pathway.³⁶⁹⁻³⁸ The classical pathway can result in formation of the membrane attack complex, resulting in cell lysis.^{38,39}

Our analysis also found significant decreases in serum exosomal levels of 6 prothrombotic proteins. FIBA, FIBB and FIBG complex to form fibrinogen, which is cleaved by thrombin into fibrin strands that polymerize to form clots during wound healing.⁴⁰ FINC and TSP-1 play important roles in ECM and clot stabilization during wound healing.^{41,42} F13A crosslinks fibrin strands to each other and to FINC to stabilize clot formation.⁴⁰

With the exception of KV302 and HV304, our analysis notably found that serum exosomal levels of the aforementioned proteins were decreased in both ACR and AMR relative to non-rejection HTx samples. These decreases could reflect the depletion of serum exosomes containing immune and hemostatic mediators due to their increased utilization by host cells in allograft rejection. For example, endothelial cell inflammation and subsequent thrombosis in graft vasculature are frequently found during rejection.^{43,44} Additionally, AMR in particular is associated with capillary microthrombi and increased complement deposition in allograft microvasculature.^{43,44}. It is therefore possible that exosomes, containing proteins necessary to sustain the immune response to the allograft, are preferentially utilized and therefore appear to be depleted in serum of patients with evidence of cardiac allograft rejection.

It is not clear how exosomes are identified and selectively taken up by cells based on their contents. It has been shown, however, that specific membrane-bound proteins such as CD63 are incorporated into exosomes and can be used as exosome markers. This suggests that there may be a process by which specific proteins may be incorporated into exosome membranes based on their contents (e.g., an inflammatory exosome possesses different surface markers than an angiogenic exosomes).¹⁷ Importantly, the incorporation of these exosome markers is independent of their cellular concentrations, which means that there is an active sorting mechanism that selects what is loaded on or into exosomes. There is some evidence that the endosomal sorting complex required for transport (ESCRT) machinery plays a role not only in exosome biogenesis but also in this process.¹⁸ Further studies are needed to investigate the role of these exosomal complement factors in cardiac allograft rejection.

Despite its shortcomings, EMB has remained the standard for cardiac allograft rejection diagnosis due to the lack of a suitable alternative.⁷ EMB carries a complication rate of approximately 6%, with almost 1% of patients experiencing potentially fatal complications such as ventricular perforation.^{5,45} Risks are especially high immediately post-transplant, when patients must undergo the procedure as often as weekly for the first month.^{8,45} Moreover, histological diagnosis and grading can be limited due to subjectivity and can significantly affect treatment.⁴⁶⁻⁴⁸ A safer, less invasive and more objective approach to diagnosis is clearly necessary and warranted.

Recent years have seen several attempts at developing a promising alternative to EMB. AlloMap (CareDx) is a commercially available test that quantifies expression of 11 genes using qPCR to determine a patient's risk of developing ACR.⁴⁹ Several clinical studies have confirmed the effectiveness of the test, which is comparable to EMB in diagnosing ACR and is even capable of delivering a diagnosis earlier than EMB.⁵⁰⁻⁵² However, the gene panel used by AlloMap is specific to ACR and cannot diagnose patients with AMR.⁸ A recent study by De Vlaminck et al. (2014) used high-throughput screening to identify circulating cell-free DNA (cfDNA) quantification as an effective and noninvasive diagnostic measure comparable to EMB.⁵³ However, only limited studies on cfDNA in allograft rejection are available, and their precise role in mediating rejection remains to be elucidated.⁵³⁻⁵⁵

We show that exosomal proteome both allow for the diagnosis of rejection and enable a deeper understanding of the intricacies of cell-cell communication during rejection. Under normal conditions, cardiomyocytes and cardiac progenitor cells have been shown to secrete exosomes containing anti-apoptotic and pro-angiogenic miRNAs, which can stimulate infarct healing when injected in vivo in mice.⁵⁶ Murine embryonic stem cells have also been shown to have the same effect in cardiac repair post-infarct.⁵⁷ Conversely, exosomal contents, particular miRNAs, can exacerbate pathological states such as cardiac hypertrophy and septic cardiomyopathy.⁵⁸ Within the field of transplantation, there is some evidence that immune cell-derived exosomes could improve graft tolerance induction when combined with immunosuppression.^{59,60} Additionally, Sigdel et al. identified 11 pro-inflammatory proteins that were increased in urine exosomes in patients undergoing renal allograft rejection compared to transplant patients without rejection.⁶¹

Our study identified 15 serum exosomal proteins that differed significantly across non-rejection, ACR and AMR groups. The exosomal proteins may be tested alone or as part of a combination panel. Our blood-based approach could present a distinct advantage over the much more invasive EMB, significantly reducing complications and patient discomfort as well as circumventing the subjectivity of histopathology. Additionally, our approach can enable the identification and early treatment of ACR and AMR cases.

In conclusion, we have demonstrated that episodes of acute cardiac allograft rejection cause significant changes in several serum exosomal proteins, probably due to increased cellular utilization and subsequent depletion from the circulation. A combination panel assay for these proteins could have strong potential as an effective and specific test for cardiac allograft rejection.

### REFERENCES

1. Heidenreich P, Albert N, Allen L, et al. Forecasting the impact of heart failure in the United States: a policy statement from the American Heart Association. Circulation: Heart Failure 2013; 6(3): 606-19.
2. McMurray J J, Pfeffer M A. Heart failure. Lancet 2005; 365(9474): 1877-89.
3. Neubauer S. The failing heart—an engine out of fuel. The New England journal of medicine 2007; 356(11): 1140-51.
4. Rosamond W, Flegal K, Furie K, et al. Heart disease and stroke statistics-2008 update: a report from the American Heart Association Statistics Committee and Stroke Statistics Subcommittee. Circulation 2008; 117(4): e25-146.
5. Saraiva F, Matos V, Goncalves L, Antunes M, Providência L. Complications of endomyocardial biopsy in heart transplant patients: a retrospective study of 2117 consecutive procedures. Transplantation Proceedings 2011; 43(5): 1908-12.
6. Stehlik J, Edwards L B, Kucheryavaya A Y, et al. The Registry of the International Society for Heart and Lung Transplantation: twenty-seventh official adult heart transplant report-2010. J Heart Lung Transplant 2010; 29(10): 1089-103.
7. Caves P K, Stinson E B, Graham A F, Billingham M E, Grehl T M, Shumway N E. Percutaneous transvenous endomyocardial biopsy. JAMA 1973; 225(3): 288-91.
8. Kittleson M M, Kobashigawa J A. Long-term care of the heart transplant recipient. Curr Opin Organ Transplant 2014; 19(5): 515-24.
9. Liu D Q, Li F F, Zhang J B, et al. Increased RIPK4 expression is associated with progression and poor prognosis in cervical squamous cell carcinoma patients. Sci Rep 2015; 5: 11955.
10. Reichard C A, Stephenson A J, Klein E A. Applying precision medicine to the active surveillance of prostate cancer. Cancer 2015.
11. Sung T Y, Kim M, Kim T Y, et al. Negative Expression of CPSF2 Predicts a Poorer Clinical Outcome in Patients with Papillary Thyroid Carcinoma. Thyroid 2015.
12. Zhang L, Zhang J, Ma Y, et al. Testicular orphan receptor 4 (TR4) is a marker for metastasis and poor prognosis in non-small cell lung cancer that drives the EMT phenotype. Lung Cancer 2015.
13. Strimbu K, Tavel J A. What are biomarkers? Curr Opin HIV AIDS 2010; 5(6): 463-6.
14. Duong Van Huyen J P, Tible M, Gay A. et al. MicroRNAs as non-invasive biomarkers of heart transplant rejection. Eur Heart J 2014; 35(45): 3194-202.
15. Qian W J, Jacobs J M, Liu T, Camp D G, 2nd, Smith R D. Advances and challenges in liquid chromatography-mass spectrometry-based proteomics profiling for clinical applications. Mol Cell Proteomics 2(006; 5(10): 1727-44.
16. Sukma Dewi I, Torngren K, GidlofO, Kornhall B, Ohman J. Altered serum miRNA profiles during acute rejection after heart transplantation: potential for non-invasive allograft surveillance. J Heart Lung Transplant 2013; 32(4): 463-6.
17. Record M, Subra C, Silvente-Poirot S, Poirot M. Exosomes as intercellular signalosomes and pharmacological effectors. Biochemical Pharmacology 2011; 81(10): 1171-82.
18. Simons M, Raposo G. Exosomes—vesicular carriers for intercellular communication. Curr Opin Cell Biol 2009; 21(4): 575-81.
19. Toro J D, Herschlik L, Waldner C, Mongini C. Emerging roles of exosomes in normal and pathological conditions: new insights for diagnosis and therapeutic applications. Frontiers in Immunology 2015; 6(203).
20. Colombo M, Moita C, van Niel G, et al. Analysis of ESCRT functions in exosome biogenesis, composition and secretion highlights the heterogeneity of extracellular vesicles. J Cell Set 2013; 126(Pt 24): 5553-65.
21. S ELA, Mager I, Breakefield X O, Wood M J. Extracellular vesicles: biology and emerging therapeutic opportunities. Nat Rev Drug Discov 2013; 12(5): 347-57.
22. Li J, Sherman-Baust C A, Tsai-Turton M, Bristow R E, Roden R B, Morin P J. Claudin-containing exosomes in the peripheral circulation of women with ovarian cancer. BMC Cancer 2009; 9: 244.
23. Nilsson J. Skog J, Nordstrand A, et al. Prostate cancer-derived urine exosomes: a novel approach to biomarkers for prostate cancer. Br J Cancer 2009; 100(10): 1603-7.
24. Thery C, Amigorena S, Raposo G, Clayton A. Isolation and characterization of exosomes from cell culture supernatants and biological fluids. Curr Protoc Cell Biol 2006; Chapter 3: Unit 3 22.
25. Lane R E, Korbie D, Anderson W, Vaidyanathan R Trau M. Analysis of exosome purification methods using a model liposome system and tunable-resistive pulse sensing. Sci Rep 2015; 5: 7639.
26. Yates J R, 3rd, Eng J K, McCormack A L, Schieltz D. Method to correlate tandem mass spectra of modified peptides to amino acid sequences in the protein database. Anal Chem 1995; 67(8): 1426-36.
27. UniProt C. Activities at the Universal Protein Resource (UniProt). Nucleic acids research 2014; 42(Database issue): D191-8.
28. Elias J E, Gygi S P. Target-decoy search strategy for increased confidence in large-scale protein identifications by mass spectrometry. Nature methods 2007; 4(3): 207-14.
29. Bhatnagar S, Shinagawa K, Castellino F J, Schorey J S. Exosomes released from macrophages infected with intracellular pathogens stimulate a proinflammatory response in vitro and in vivo. Blood 2007; 110(9): 3234-44.
30. Robbins P D, Morelli A E. Regulation of immune responses by extracellular vesicles. Nat Rev Immunol 2014; 14(3): 195-208.
31. MacKenzie A, Wilson H L, Kiss-Toth E, Dower S K, North R A, Surprenant A. Rapid secretion of interleukin-Ibeta by microvesicle shedding. Immunity 2001; 15(5): 825-35.
32. Pizzirani C, Ferrari D, Chiozzi P, et al. Stimulation of P2 receptors causes release of IL-1beta-loaded microvesicles from human dendritic cells. Blood 2007; 109(9): 3856-64.
33. Monajemi H, Fontijn R D, Pannekoek H, Horrevoets A J. The apolipoprotein L gene cluster has emerged recently in evolution and is expressed in human vascular tissue. Genomics 2002; 79(4): 539-46.
34. Wan G, Zhaorigetu S, Liu Z, Kaini R, Jiang Z, Hu C A. Apolipoprotein L1, a novel Bcl-2 homology domain 3-only lipid-binding protein, induces autophagic cell death. J Biol Chem 2008; 283(31): 21540-9.
35. Zhaorigetu S, Wan G, Kaini R, Jiang Z. Hu C A. ApoL1, a BH3-only lipid-binding protein, induces autophagic cell death. Autophagy 2008; 4(8): 1079-82.
36. Arlaud G J, Gaboriaud C, Thielens N M, et al. Structural biology of C1: dissection of a complex molecular machinery. Immunol Rev 2001; 180: 136-45.
37. Cooper N R. The classical complement pathway: activation and regulation of the first complement component. Adv Immunol 1985; 37: 151-216.
38. Merle N S, Church S E, Fremeaux-Bacchi V, Roumenina L T. Complement System Part I—Molecular Mechanisms of Activation and Regulation. Front Immunol 2015; 6: 262.
39. Merle N S, Noe R, Halbwachs-Mecarelli L, Fremeaux-Bacchi V, Roumenina L T. Complement System Part I I: Role in Immunity. Front Immunol 2015; 6: 257.
40. Hoppe B. Fibrinogen and factor XIII at the intersection of coagulation, fibrinolysis and inflammation. Thromb Haemost 2014; 112(4): 649-58.
41. Labat-Robert J. Cell-Matrix interactions, the role of fibronectin and integrins. A survey. Pathol Biol (Paris) 2012; 60(1): 15-9.
42. Krishna S M, Golledge J. The role of thrombospondin-1 in cardiovascular health and pathology. Int J Cardiol 2013; 168(2): 692-706.
43. Al-Lamki R S, Bradley J R, Pober J S. Endothelial cells in allograft rejection. Transplantation 2008; 86(10): 1340-8.
44. Fishbein G A, Fishbein M C. Morphologic and immunohistochemical findings in antibody-mediated rejection of the cardiac allograft. Hum Immunol 2012; 73(12): 1213-7.
45. From A M, Maleszewski J J, Rihal C S. Current status of endomyocardial biopsy. Mayo Clin Proc 2011; 86(11): 1095-102.
46. Angelini A, Andersen C B, Bartoloni G, et al. A web-based pilot study of inter-pathologist reproducibility using the ISHLT 2004 working formulation for biopsy diagnosis of cardiac allograft rejection: the European experience. J Heart Lung Transplant 2011; 30(11): 1214-20.
47. Winters G L, McManus B M. Consistencies and controversies in the application of the International Society for Heart and Lung Transplantation working formulation for heart transplant biopsy specimens. Rapamycin Cardiac Rejection Treatment Trial Pathologists. J Heart Lung Transplant 1996; 15(7): 728-35.
48. Yang H M, Lai C K, Gjertson D W, et al. Has the 2004 revision of the International Society of Heart and Lung Transplantation grading system improved the reproducibility of the diagnosis and grading of cardiac transplant rejection? Cardiovasc Pathol 2009; 18(4): 198-204.
49. Dedrick R L. Understanding gene expression patterns in immune-mediated disorders. J Immunotoxicol 2007; 4(3): 201-7.
50. Deng M C, Eisen H J, Mehra M R, et al. Noninvasive discrimination of rejection in cardiac allograft recipients using gene expression profiling. Am J Transplant 2006; 6(1): 150-60.
51. Pham M X, Teuteberg J J, Kfoury A G, et al. Gene-expression profiling for rejection surveillance after cardiac transplantation. N Engl J Med 2010; 362(20): 1890-900.
52. Sarwal M, Sigdel T. A common blood gene assay predates clinical and histological rejection in kidney and heart allografts. Clin Transpl 2013: 241-7.
53. De Vlaminck 1, Valantine H A, Snyder T M, et al. Circulating cell-free DNA enables noninvasive diagnosis of heart transplant rejection. Sci Transl Med 2014; 6(241): 241ra77.
54. Daly K P. Circulating donor-derived cell-free DNA: a true biomarker for cardiac allograft rejection? Ann Transl Med 2015; 3(4): 47.
55. Hidestrand M, Tomita-Mitchell A, Hidestrand P M, et al. Highly sensitive noninvasive cardiac transplant rejection monitoring using targeted quantification of donor-specific cell-free deoxyribonucleic acid. J Am Coll Cardiol 2014; 63(12): 1224-6.
56. Cervio E, Barile L, Moccetti T, Vassalli G. Exosomes for Intramyocardial Intercellular Communication. Stem Cells Int 2015; 2015; 482171.
57. Khan M, Nickoloff E, Abramova T, et al. Embryonic Stem Cell-Derived Exosomes Promote Endogenous Repair Mechanisms and Enhance Cardiac Function Following Myocardial Infarction. Circ Res 2015; 117(1): 52-64.
58. Ailawadi S, Wang X, Gu H, Fan G C. Pathologic function and therapeutic potential of exosomes in cardiovascular disease. Biochim Biophys Acta 2015; 1852(1): 1-11.
59. Agarwal A, Fanelli O, Letizia M, et al. Regulatory T cell-derived exosomes: possible therapeutic and diagnostic tools in transplantation. Front Immunol 2014; 5: 555.

60. Li X, Li J J, Yang J Y, et al. Tolerance induction by exosomes from immature dendritic cells and rapamycin in a mouse cardiac allograft model. PLoS One 2012; 7(8): e44045.
61. Sigdel T K, Ng Y W, Lee S, et al. Perturbations in the urinary exosome in transplant rejection. Front Med (Lausanne) 2014; 1: 57.
62. Ratajczak J, Wysoczynski M, Hayek F, Janowska-Wieczorek A, Ratajczak M Z. Membrane-derived microvesicles: important and underappreciated mediators of cell-to-cell communication. Leukemia 2006; 20(9): 1487-95.
63. Lubec G, Afjehi-Sadat L. Limitations and pitfalls in protein identification by mass spectrometry. Chem Rev 2007; 107(8): 3568-84.

All references cited herein are incorporated by reference to the same extent as if each individual publication, database entry (e.g. UniProt, Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. UniProt, Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

What is claimed is:

1. A method for detecting transplant rejection in a subject who has received a transplant or assessing the subject's risk of transplant rejection, the method comprising the steps of:
S (a) obtaining a sample from the subject;
(b) isolating exosomes from the sample;
(c) determining level of one or more exosomal polypeptides in the exosomes;
(d) comparing the level obtained in step (c) with the level of the one or more exosomal polypeptide in a control sample; and
(e) diagnosing that the subject has transplant rejection or an increased risk of transplant rejection, if the level of at least one exosomal polypeptide obtained in step (c) increases or decreases by at least 10% compared to its level in the control sample.

2. A method of treating a subject with transplant rejection or an increased risk of transplant rejection, the method comprising the steps of:
(a) obtaining a sample from the subject;
(b) isolating exosomes from the sample;
(c) determining level of one or more exosomal polypeptides in the exosomes;
(d) comparing the level obtained in step (c) with the level of the one or more exosomal polypeptides in a control sample; and
(e) treating the subject for transplant rejection or an increased risk of transplant rejection, if the level of at least one exosomal polypeptide obtained in step (c) increases or decreases by at least 10% compared to its level in the control sample.

3. The method of claim 2, wherein in step (e) at least one immunosuppressant is administered to the subject.

4. The method of claim 1 or 2, wherein the exosomal polypeptide is selected from the group consisting of C1QA, C1R, KV302, HV304, HV315, FIBA, FIBB, FIBG, FINC, F13A, TSP1, FRMPD1, ITIH1, APOL1, ACTB, and combinations thereof.

5. The method of claim 1 or 2, wherein the exosomal polypeptide is selected from the group consisting of C1QA, FINC, KV302, HV304, and combinations thereof.

6. The method of claim 1 or 2, wherein the exosomal polypeptide is selected from the group consisting of LV101, IGJ, STK36, L1CAM, KV302, ITIH2, PLMN, PON1, C1RL, KV303, KV1A1, B7ZKJS, FIBG, FIBB, CO5, LV102, A2AP, and combinations thereof.

7. The method of claim 1 or 2, wherein the exosomal polypeptide is selected from the group consisting of LV02, FIBG, FIBB, FIBA, ACTB, ECM1, F13A, C1R, FINC, TSP1, TNNC1, FSVV04, STK36, IGJ, TOP2A, LV101, TRIPB, GK, LICAM, PON1, C1RL, ITIH2, KLKB1, HV315, APOL1, GELS, IGHD, ITIH1, FRMPD1, PLMN, KV302, FSW6P5, C9JMH6, B7ZKJS, KV1A1, F5H7E1, A1AG1, A2AP, HV304, GSJLSS, E9PBC5, QSVY30, Q5T9S5, C9JA05, F5H4W9, and combinations thereof.

8. The method of claim 1 or 2, wherein the exosomal polypeptide is selected from the group consisting of fibronectin, IGHM, LV101, HBB, and combinations thereof.

9. A method for detecting transplant rejection in a subject who has received a transplant or assessing the subject's risk of transplant rejection, the method comprising the steps of:
(a) obtaining a sample from the subject;
(b) determining in the sample the level of one or more polypeptides selected from the group consisting of C1QA, C1R, KV302, HV304, HV315, FIBA, FIBB, FIBG, FINC, F13A, TSP1, FRMPD1, IT1H1, APOL1 and ACTB;
(c) comparing the level obtained in step (b) with the level of the one or more polypeptides in a control sample; and
(d) diagnosing that the subject has transplant rejection or an increased risk of transplant rejection, if the level of at least one polypeptide obtained in step (b) increases or decreases by at least 10% compared to its level in the control sample.

10. A method of treating a subject with transplant rejection or an increased risk of transplant rejection, the method comprising the steps of:
(a) obtaining a sample from the subject;
(b) determining in the sample level of one or more polypeptide selected from the group consisting of C1QA, C1R, KV302, HV304, HV315, FIBA, FIBB, FIBG, FINC, F13A, TSP1, FRMPD1, IT1H1, APOL1 and ACTB;
(c) comparing the level obtained in step (b) with the level of the one or more polypeptide in a control sample; and
(d) treating the subject for transplant rejection or an increased risk of transplant rejection, if the level of at least one polypeptide obtained in step (b) increases or decreases by at least 10% compared to its level in the control sample.

11. The method of claim 10, wherein in step (d) at least one immunosuppressant is administered to the subject.

12. The method of claim 9 or 10, wherein the polypeptide is an exosomal protein.

13. The method of claim 9 or 10, wherein after step (a) exosomes are isolated from the sample, and in step (b) the level of the at least one polypeptide in the exosomes is determined.

14. The method of any of claims 1, 2, 9 or 10, wherein the increase or decrease in the level of the at least one polypeptide is at least 50%.

15. The method of any of claims 1, 2, 9 or 10, wherein the increase or decrease in the level of the at least one polypeptide is at least 70%.

16. The method of any of claims 1, 2, 9 or 10, wherein the increase or decrease in the level of the at least one polypeptide is at least 90%.

17. The method of any of claims 1, 2, 9 or 10, wherein the increase or decrease in the level of the at least one polypeptide ranges from about 20% W to about 90%.

18. The method of any of claims 1, 2, 9 or 10, wherein the increase or decrease in the level of the at least one polypeptide ranges from about 50% to about 100%.

19. The method of any of claims 1, 2, 9 or 10, wherein the sample is a plasma, serum or blood sample.

20. The method of any of claims 1, 2, 9 or 10, wherein the transplant is a heart transplant, a kidney transplant, a lung transplant, a liver transplant, a pancreas transplant, a bone marrow transplant, a portion thereof, or a combination thereof.

21. The method of any of claims 1, 2, 9 or 10, wherein the transplant is a tissue transplant.

22. The method of any of claims 1, 2, 9 or 10, wherein the control sample is from a healthy subject or a plurality of healthy subjects.

23. The method of any of claims 1, 2, 9 or 10, wherein the control sample is from a subject who has received a transplant without rejection or from a plurality of subjects who have received a transplant without rejection.

24. The method of any of claims 1, 2, 9 or 10, wherein the transplant rejection comprises acute cellular rejection (ACR) and/or antibody-mediated rejection (AMR).

25. The method of any of claims 1, 2, 9 or 10, wherein the transplant rejection is hyperacute rejection.

26. The method of any of claims 1, 2, 9 or 10, wherein the transplant rejection is acute rejection.

27. The method of any of claims 1, 2, 9 or 10, wherein the transplant rejection is chronic transplant rejection.

28. The method of any of the preceding claims, wherein the subject is human.

29. The method of any of the preceding claims, wherein the subject's existing immunosuppressive regimen is modified or maintained.

30. The method of any of the preceding claims, wherein the level of the one or more polypeptides is determined by mass spectrometry (MS).

31. The method of any of claims 1, 2, 9 or 10, wherein the level of the one or more polypeptides is determined by enzyme-linked immunosorbent assay (ELISA).

32. A kit comprising:
antibodies or fragments thereof that specifically bind to one or more exosomal polypeptides in a plasma or serum sample from a subject who has received a transplant; and
instructions for measuring the one or more exosomal polypeptides for diagnosing transplant rejection in the subject or assessing the subject's risk of transplant rejection.
